(19) United States
(12) Patent Application Publication
(10) Pub. No.: US 2017/0296499 A1
(43) Pub. Date: Oct. 19, 2017

(54) COMBINATIONAL COMPOSITIONS AND METHODS OF USE THEREOF

(71) Applicant: Michael Lucey, Mesa, AZ (US)

(72) Inventor: Michael Lucey, Mesa, AZ (US)

(21) Appl. No.: 15/098,997

(22) Filed: Apr. 14, 2016

(51) Int. Cl. A61K 31/202; A61K 36/68; A61K 31/593; A61K 9/00; A61K 31/4375; A61K 31/375; A61K 45/06; A61K 35/60

(52) U.S. Cl. CPC A61K 31/202; A61K 45/06; A61K 36/68; A61K 35/60; A61K 9/0053; A61K 31/4375; A61K 31/375; A61K 31/593

(57) ABSTRACT

Disclosed are compositions and methods for the treatment of a disease or disorder comprising i) at least one fatty acid, and ii) at least one cholesterol lowering compound. Compositions and methods of the disclosure reduce total cholesterol and maintain total cholesterol homeostasis.

### FIELD OF THE DISCLOSURE

The disclosure is directed towards the field of medicine and, more specifically, the use of a combination of compounds to treat disease and regulate cholesterol levels.

### BACKGROUND

There is no cure for diseases and disorders based on autoimmune and inflammatory mechanisms. Existing therapies suppress the immune system to such an extent that the treatments expose patients to additional disorders or require additional therapy for the side effects of the first treatment. Many medicaments prescribed for a disease not related to either autoimmunity or inflammation can induce these conditions as a side effect. There is a long felt yet unmet need in the art for an efficacious treatment for autoimmune and inflammatory conditions that do not inhibit the ability of the immune system to function but rather resets the level of activity of the immune system back to a healthy homeostatic level.

### SUMMARY

The compositions and methods of the disclosure reduce autoimmune and inflammatory activity in a subject in need thereof by establishing an immunological homeostasis through the administration of a combination of a fatty acid and a cholesterol lowering compound (e.g. a statin).

The disclosure provides a pharmaceutical composition for the treatment of a disease or disorder comprising i) at least one fatty acid, and ii) at least one cholesterol lowering compound.

The disclosure provides a pharmaceutical composition for reducing total cholesterol and maintaining total cholesterol homeostasis comprising i) at least one fatty acid, and ii) at least one cholesterol lowering compound. In certain embodiments, maintaining total cholesterol homeostasis comprises maintaining a total cholesterol percent relative range of about 15%, of about 10%, or of about 5% between a maximum total cholesterol level and a minimum total cholesterol level. In certain embodiments, maintaining total cholesterol homeostasis comprises maintaining a total cholesterol range of about 5 mg/dL to about 25 mg/dL or of about 10 mg/dL to about 15 mg/dL between a maximum total cholesterol level and a minimum total cholesterol level. In certain embodiments, a maximum total cholesterol level and a minimum total cholesterol level are maintained below 200 mg/dL, below 175 mg/dL, or below 150 mg/dL. In certain embodiments, total cholesterol is reduced at least 5%, at least 10%, at least 15%, at least 20% or at least 40%.

Pharmaceutical compositions of the disclosure comprise at least one fatty acid. Exemplary fatty acids include, but are not limited to, a fatty oil, a fatty acid ethyl ester, a fatty acid triglyceride, a saturated acid, an oil, an ester or a triglyceride, or a combination thereof. Exemplary fatty acids may further include, but are not limited to, an Omega-3 fatty acid, an Omega-6 fatty acid, an Omega-9 fatty acid, or a derivative thereof.

Pharmaceutical compositions of the disclosure comprise at least one cholesterol lowering compound. Exemplary cholesterol lowering compounds include, but are not limited to, an HMG-CoA reductase inhibitor or statin, Ezetimibe, a fibrate, a carboxylic acid, Benezafibrate, Ciprofibrate, Gemfbroizil, Fenofibrate, Clinofibrate, niacin, bile acid sequestrants, Colestipol, Cholestyramine, Endur-Acin, Colesevelam, a PCSK9 enzyme inhibitor, or any combination thereof. In certain embodiments, the at least one HMG-CoA reductase inhibitor or statin may be Atorvastatin, Fluvastatin, Lovastatin, Pitavastatin, Pravastatin, Rosuvastatin, Simvastatin, or any combination thereof.

Pharmaceutical compositions of the disclosure may comprise at least one vitamin or hormone. Exemplary vitamins include, but are not limited to, vitamin B, vitamin C or vitamin D, or a combination thereof. In certain embodiments, the at least one vitamin D may be vitamin D₂ or vitamin D₃. In certain embodiments, the at least one vitamin C may be ascorbate or ascorbic acid.

Pharmaceutical compositions of the disclosure may comprise at least one organic compound, herb or derivative thereof. Exemplary organic compounds, herbs or derivatives thereof include, but are not limited to, bacopa, vinpocetine, an alkaloid, reserpine, reserpinine, akuammicine, majdine, vinerine, ervine, vineridine, tombozine, vincananine, vincanidine, vincamore, apovincamine, vincamore, apovincamine, or vincaminol.

Pharmaceutical compositions of the disclosure may be used in the treatment of a disease or disorder including, but not limited to, an autoimmune disease, an inflammatory disorder, a neurodegenerative disorder, a bacterial infection or a viral infection. In certain embodiments, the disease or disorder may be psoriasis, keratosis, spongy gum and bleeding gum disease, atherosclerosis, heart disease, myopathy, neuropathy, common cold, myositis, arthritis, dementia, Parkinson's disease, Alzheimer's disease. In certain embodiments, the keratosis may be actinic, pilaris or seborrheic. In certain embodiments, the pharmaceutical composition may be used in the treatment of the common cold. As used herein, the “common cold” comprises a viral infection of a portion of the upper respiratory tract.

The disclosure provides a pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA) and at least 10 mg of a cholesterol lowering composition. In certain embodiments, the composition may be formulated for daily oral administration for use in the treatment of psoriasis.

The disclosure provides a pharmaceutical composition comprising at least 1000 mg of a fish oil, at least 667 mg of a EPA, at least 333 mg of a DHA and at least 10 mg of a cholesterol lowering composition. In certain embodiments, the composition may be formulated for daily oral administration for use in the treatment of keratosis, atherosclerosis, heart disease, or any combination thereof.

The disclosure provides a pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA), at least 10 mg of a cholesterol lowering composition and at least 1000 mg of vitamin C. In certain embodiments, the composition may be formulated for daily oral administration for use in the treatment of spongy gum and bleeding gum disease.

The disclosure provides a pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA), at least 10 mg of a cholesterol lowering composition and at least 3000 mg of vitamin C. In certain embodiments, the composition may be formulated for daily oral administration for use in the treatment of a bacterial infection, viral infection or the common cold.

The disclosure provides a pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA), at least 10 mg of a cholesterol lowering composition and at least 2000 IU of vitamin D. In certain embodiments, the composition may be formulated for daily oral administration for use in the treatment of myopathy or neuropathy.

The disclosure provides a pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA), at least 10 mg of a cholesterol lowering composition, and at least 250 mg of bacopa. In certain embodiments, the composition may further comprise at least 10 mg of vinpocetine. In certain embodiments, the composition may be formulated for daily oral administration for use in the treatment of dementia, Parkinson's disease or Alzheimer's disease.

The disclosure provides a method for the treatment of a disease or disorder in a subject in need thereof comprising administering a therapeutically effective amount of a pharmaceutical composition comprising i) at least one fatty acid, and ii) at least one cholesterol lowering compound, such that said disease or disorder is treated.

The disclosure provides a method for reducing total cholesterol and maintaining total cholesterol homeostasis in a subject in need thereof comprising administering a therapeutically effective amount of a pharmaceutical composition comprising i) at least one fatty acid, and ii) at least one cholesterol lowering compound, such that total cholesterol is reduced and total cholesterol homeostasis is maintained. In certain embodiments, maintaining total cholesterol homeostasis comprises maintaining a total cholesterol percent relative range of about 15%, of about 10%, or of about 5% between a maximum total cholesterol level and a minimum total cholesterol level while the pharmaceutical composition is administered. In certain embodiments, maintaining total cholesterol homeostasis comprises maintaining a total cholesterol range of about 5 mg/dL to about 25 mg/dL or of about 10 mg/dL to about 15 mg/dL between a maximum total cholesterol level and a minimum total cholesterol level while the pharmaceutical composition is administered. In certain embodiments, a maximum total cholesterol level and a minimum total cholesterol level are maintained below 200 mg/dL, below 175 mg/dL, or below 150 mg/dL while the pharmaceutical composition is administered. In certain embodiments, total cholesterol is reduced at least 5%, at least 10%, at least 15%, at least 20% or at least 40%. In certain embodiments, reducing total cholesterol levels comprises reducing triglyceride levels.

In certain embodiments of the methods of the disclosure, the pharmaceutical composition may comprise at least one fatty acid. Exemplary fatty acids include, but are not limited to, a fatty oil, a fatty acid ethyl ester, a fatty acid triglyceride, a saturated acid, an oil, an ester or a triglyceride, or a combination thereof. Exemplary fatty acids may further include, but are not limited to, an Omega-3 fatty acid, an Omega-6 fatty acid, an Omega-9 fatty acid, or a derivative thereof.

In certain embodiments of the methods of the disclosure, the pharmaceutical composition may comprise at least one cholesterol lowering compound. Exemplary cholesterol lowering compounds may include, but are not limited to, an HMG-CoA reductase inhibitor or statin, Ezetimibe, a fibrate, a carboxylic acid, Benezafibrate, Ciprofibrate, Gemfbroizil, Fenofibrate, Clinofibrate, niacin, bile acid sequestrants, Colestipol, Cholestyramine, Endur-Acin, Colesevelam, a PCSK9 enzyme inhibitor, or any combination thereof. In certain embodiments, the at least one HMG-CoA reductase inhibitor or statin may be Atorvastatin, Fluvastatin, Lovastatin, Pitavastatin, Pravastatin, Rosuvastatin, Simvastatin, or any combination thereof.

In certain embodiments of the methods of the disclosure, the pharmaceutical composition may comprise at least one vitamin or hormone. The at least one vitamin may include vitamin B, vitamin C, vitamin D or a combination thereof. In certain embodiments, the vitamin D may be vitamin D₂ or vitamin D₃. In certain embodiments, the vitamin C is ascorbate or ascorbic acid.

In certain embodiments, the methods of the disclosure include administering a pharmaceutical composition and at least one organic compound, herb or derivative thereof. The at least one organic compound, herb or derivative thereof may include, but is not limited to, bacopa, vinpocetine, an alkaloid, reserpine, reserpinine, akuammicine, majdine, vinerine, ervine, vineridine, tombozine, vincananine, vincanidine, vincamore, apovincamine, vincamore, apovincamine, and vincaminol.

Compositions and methods of the disclosure may be used in the treatment of a disease or disorder, including, but not limited to, an autoimmune disease, an inflammatory disorder, a neurodegenerative disorder, a bacterial infection or a viral infection. In certain embodiments, the disease or disorder may be psoriasis, keratosis, spongy gum and bleeding gum disease, atherosclerosis, heart disease, myopathy, neuropathy, common cold, myositis, arthritis, dementia, Parkinson's disease, Alzheimer's disease. In certain embodiments, the keratosis may be actinic, pilaris or seborrheic.

The disclosure provides a method for treating psoriasis in a subject in need thereof comprising administering a therapeutically effective amount of a pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA) and at least 10 mg of a cholesterol lowering composition.

According to the compositions and methods of the disclosure, pharmaceutical compositions may be formulated for oral delivery.

According to the compositions and methods of the disclosure, pharmaceutical compositions may be administered daily or weekly.

The disclosure provides a method for treating keratosis, atherosclerosis, heart disease, or any combination thereof, in a subject in need thereof comprising administering a therapeutically effective amount of a pharmaceutical composition comprising at least 1000 mg of a fish oil, at least 667 mg of a EPA, at least 333 mg of a DHA and at least 10 mg of a cholesterol lowering composition. In certain embodiments, the pharmaceutical composition may be formulated for oral delivery. In certain embodiments, the pharmaceutical composition may be administered daily or weekly.

The disclosure provides a method for treating spongy gum and bleeding gum disease in a subject in need thereof comprising administering a therapeutically effective amount of a pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA), at least 10 mg of a cholesterol lowering composition and at least 1000 mg of vitamin C. In certain embodiments, the pharmaceutical composition may be formulated for oral delivery. In certain embodiments, the pharmaceutical composition may be administered daily or weekly.

The disclosure provides a method of treating a bacterial infection, viral infection or the common cold in a subject in need thereof comprising administering a therapeutically effective amount of a pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA), at least 10 mg of a cholesterol lowering composition and at least 3000 mg of vitamin C. In certain embodiments, the pharmaceutical composition may be formulated for oral delivery. In certain embodiments, the pharmaceutical composition may be administered daily or weekly.

The disclosure provides a method of treating myopathy or neuropathy in a subject in need thereof comprising administering a therapeutically effective amount of a pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA), at least 10 mg of a cholesterol lowering composition and at least 2000 IU of vitamin D. In certain embodiments, the pharmaceutical composition may be formulated for oral delivery. In certain embodiments, the pharmaceutical composition may be administered daily or weekly.

The disclosure provides a method of treating dementia, Parkinson's disease or Alzheimer's disease in a subject in need thereof comprising administering a therapeutically effective amount of a pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA), at least 10 mg of a cholesterol lowering composition, and at least 250 mg of bacopa. In certain embodiments, the pharmaceutical composition may be formulated for oral delivery. In certain embodiments, the pharmaceutical composition may be administered daily or weekly.

### BRIEF DESCRIPTION OF THE DRAWINGS

FIG. 1 is a graph depicting total cholesterol (mg/dL) as a function of time for Patient A (data points provided in Table 2) over first time period (November 2001 to July 2011).

FIG. 2 is a graph depicting total cholesterol (mg/dL) as a function of time for Patient A (data points provided in Table 2) over second time period (August 2011 to August 2015).

FIG. 3 is a graph depicting total cholesterol (mg/dL) as a function of time for Patient B (data points provided in Table 3) over first time period (November 1998 to May 2013).

FIG. 4 is a graph depicting total cholesterol (mg/dL) as a function of time for Patient B (data points provided in Table 3) over second time period (August 2013 to May 2015).

FIG. 5 is a schematic diagram depicting an exemplary timeline for administration of a composition of the disclosure with exemplary data points following collection of a sample and measurement of total cholesterol from, for example, circulating blood.

### DETAILED DESCRIPTION

The disclosure provides a pharmaceutical composition for the treatment of a disease or disorder comprising i) at least one fatty acid, and ii) at least one cholesterol lowering compound. Compositions and methods of the disclosure reduce total cholesterol and maintain total cholesterol homeostasis.

Some cholesterol lowering medicaments may establish a homeostatic cholesterol level. For example the combination of Lipitor and TriCor (fenofibrate) or Vytorin (a combination of Zetia and Simvastatin) as a monotherapy may establish a homeostatic cholesterol level. In contrast, neither Lipitor nor Zetia, when administered as a monotherapy, will establish a homeostatic cholesterol level. Both Lipitor and Zetia need an Omega-3 fish oil to establish a homeostatic cholesterol level, as illustrated in Patient A's Experiments 5-15.

Importantly, cholesterol lowering agents administered as a monotherapy will not treat autoimmune or inflammatory diseases. Cholesterol lowering agents require a coinitiator, including, but not limited to, a fatty acid, a vitamin, a hormone, a flavanoid, a carotenoid, a retinoid, a mineral, or any combination thereof. Likewise, the co-initiator requires a statin, a cholesterol lowering agent, a lipoprotein lowering agent, a triglyceride lowering agent (e.g. statin(s), fibrate(s), PCSK9, monoclonal antibody inhibitors, ApoA-1 milano, succinofuco (AG1-1067), and Apoprotein-B inhibitor Mipomersen) or any combination thereof.

For example, Omega-3 fatty acids (triglyceride or ethyl ester) or vitamin D may serve as a co-initiator with a statin. This combination of a fatty acid, a vitamin and a statin may be used to efficaciously treat autoimmune and/or inflammatory diseases, including, but not limited to, myopathy, myositis, psoriasis, keratosis, atherosclerosis, neuropathy and other related diseases such as neurodegenerative diseases.

Compositions and methods of the disclosure may be used to treat a number of conditions having a common underlying feature of increased or unregulated fatty acids and/or cholesterol levels, either as result of the disease or disorder itself, or as a side effect of a treatment for that disease or disorder. The compositions and methods of the disclosure establish a homeostatic cholesterol level that is independent from the absolute level (or, magnitude) of the homeostatic cholesterol value. Thus, the homeostatic cholesterol level is described as a relative level compared to an initial level present either during a disease state or as a consequence of treatment of an underlying disease with another therapy.

Compositions of the disclosure may comprise one or more of a fatty acid, a fat (unsaturated or saturated fat), a vitamin, a flavanoid, a carotenoid, a retinoid, or a hormone in combination with a statin. Compositions of the disclosure may be used to treat any autoimmune or inflammatory disease.

Many autoimmune diseases are MHC-Class 1 mediated diseases. Statins repress T-lymphocyte activation, however, statins are specific for MHC-Class II and do not involve MHC-Class 1 expression. Cholesterol lowering medicaments have little or no direct association with autoimmune diseases or other diseases. Thus, treatment with a statin alone would not be efficacious to treat an autoimmune or inflammatory disease.

Thus, the compositions and methods provide a combination of a cholesterol-lowering medicament (e.g. a statin) with a fatty acid to treat disease with unexpectedly superior efficacy, to reduce cholesterol levels and to establish a healthy homeostatic cholesterol level. Unexpectedly, the compositions and methods of the disclosure are efficacious for treating autoimmune and inflammatory disorders.

Compositions and methods of the disclosure may include any statin, and, therefore, are not reliant on the use of any one particular statin.

Provided herein is a description of the treatment of eight diseases by the compositions and methods of the invention that are further illustrated by working examples.

### Psoriasis

Psoriasis is a common autoimmune disease of the skin characterized by scaly patches, papules and plaques. There are 5 prevalent forms of psoriasis: plaque, pustular, guttate, erythrodermic and invesse. Psoriasis may occur over small areas of the body or over the entire body. Areas of the more common plaque Psoriases are small (e.g. 0.5 in. in diameter) or large (e.g. 1.5-2.0 in. in diameter) patches of skin characterized by a silvery-white epidermal cell morphology.

In a subject suffering from psoriasis, the subject's immune system attacks skin cells because the epidermal cells of the skin cells are recognized, improperly, as a rapidly growing pathogen (similar to the way the immune system would recognize cancer). Consequently, psoriasis may be considered an autoimmune disease.

A subject having psoriasis may also have one or more mutations in the PSOR1 gene. This PSOR1 gene is located on chromosome 6 in the major histocompatibility complex (MHC). The MHC is a complex responsible in part for distinguishing self-antigens from non-self-antigens and preventing the development of autoimmune conditions. Psoriasis vulgaris may be associated with additional mutations in human leukocyte antigen (HLA)-C variant, including HLA-CW6. Psoriasis may be associated with an increased abundance of CD8+ T cells, HLA-CW6, IL-12b, IL-23b, TNF alpha and NF-KB.

### Keratosis

Keratosis often presents as an excessive accumulation of keratin on the epidermis or mucous membranes. Some of the more common types of keratosis include actinic, pilaris and seborrheic keratosis. Actinic keratosis is a precancerous skin condition. Actinic keratosis is characterized by usually thick, scaly and rough patches of skin. If untreated, actinic keratosis cells may transform into squamous-cell carcinoma. Keratosis pilaris is characterized by an excess of keratin that surrounds the hair follicles in the pore. The entrapped hair continues to grow causing a slightly red raised bump. Seborrheic keratosis is a non-cancerous growth of discolored skin that usually appears brown, black or light.

### Spongy Gum and Bleeding Gum Disease

Spongy gum and bleeding gum disease are associated with gingivitis and subsequent periodontal disease. Both diseases are associated with bacterial gum infections. Without treatment, the bacterial gum infections may destroy gum tissue and auxiliary bone.

Gingivitis is a minor bacterial gum inflammation that causes the gums to be soft and bleed. As gingivitis progresses it advances to periodontitis, a severe inflammation around the tooth. Over time the gums pull away from the teeth and form pockets that become infected. If the subject's immune system cannot eliminate the bacterial infection, the gum tissue and bone surrounding the tooth or teeth may be damaged or destroyed.

Patient A suffered severe spongy gum and bleeding gum disease, because he was taking both blood thinning and high blood pressure medication. The compositions and methods of the disclosure may be used to treat the severe spongy gum and bleeding gum disease that results from taking both blood thinning and high blood pressure medications.

### Myopathy

Myopathy is term used to describe a wide variety of muscular conditions. Myopathies may be inherited or acquired. Inherited myopathies include dystrophies, myotonic, congenital myopathies, mitochondrial myopathy, familial periodic paralysis, and inflammatory and metabolic myopathies. Acquired myopathies include substance induced myopathy, glucocorticoid myopathy, dermatomyositis, inclusion body myositis, polymyositis, myositis ossificans, rhabdomyolysis and myoglobinurias.

Statin induced myopathy (i.e. an acquired myopathy) often takes one of three forms: myalgia, myositis and rhabdomyolysis. Myalgia, or muscle pain, is a symptom of many diseases. Myositis, or muscle inflammation, may result in an elevation of creatine kinase; a muscle enzyme found in the blood. Rhabdomyolysis is condition in which damaged muscle (skeletal striated muscle) breaks down. Subjects having rhabdomyolysis often have muscle protein in their bloodstream that may cause severe kidney damage and eventual death.

The pain associated with myositis (myopathy) and neuropathy (e.g. nerve decay, particularly in the context of gum disease) may be treated with a composition of the disclosure comprising fish oil, Vitamin D₃ (a hormone) and a statin. When administered with fish oil, Vitamin D, in part, also controls the amount of cholesterol manufactured in the liver without negatively influencing the concentration of active statins.

### Atherosclerosis

Cardiovascular disease is the number one cause of death worldwide. Atherosclerosis (i.e., a hardening of the arteries that happens within blood vessels) is often caused by the oxidation of unsaturated fats in the bloodstream. Unsaturated fats cannot pass through the endothelial cell lining of the arterial and venial cell walls; however, oxidized fats can transport themselves across the endothelial cell membrane. The transport of oxidized fats across the walls of the blood vessels triggers an autoimmune reaction in which the subject's immune system recognizes the oxidized fat as a pathogen and attacks it. Non-oxidized fats and non-oxidized cholesterols do not cause heart disease.

Oxidized fats and oxidized cholesterol attract white blood cells called T-lymphocytes and macrophages. As white blood cells ingest the oxidized fats and cholesterol, they form the plaque that is associated with atherosclerosis, stroke and heart attacks. Macrophages form foam cells that release harmful molecules into the bloodstream. Foam cells are found in atherosclerosis and heart disease.

Peroxides and free radicals oxidize fat and cholesterol, inducing oxidative stress. Oxidative stress damages any and all components of a cell, including, but not limited to, proteins, lipids and DNA.

Atherosclerosis is characterized by the build-up of stable and unstable plaque. A hard fibrous sheath may encase the plaque formed by the autoimmune reaction. When the body successfully encases autoimmune reaction, the plaque formed is called stable plaque. When the body fails to encapsulate the autoimmune reaction (i.e. the formation of an unstable plaque), the unstable plaque may break loose. Consequently, the inflammatory cells from the reaction spread within the blood causing the blood to coagulate. The coagulation is called a blood clot. Blood clots that block the flow of blood through a blood vessel may cause a heart attack.

The combination of Omega-3 oils (fatty acids) and statin(s) stabilize the cholesterol in lipoproteins (fat deposits). Moreover, Omega-3 oils and the associated high concentration of EPA, DHA and DPA are responsible for the vasodilatory effects found in blood vessels. As a result the blood vessels widen; thereby, relaxing the smooth muscles and lowering the blood pressure.

By preventing the autoimmune response that underlies the development of atherosclerosis, the compositions and methods of the disclosure lower the risk of atherosclerosis and lower the risk of future heart attacks and strokes.

### Neuropathy

Neuropathy is a term describing damage or disease of the nerves. Neuropathy is used more often to describe damage or disease of the peripheral nervous system (PNS) as opposed to the central nervous system (CNS). Neuropathy may be caused by systemic disease, medicaments, injury, inflammation, autoimmune diseases, bacterial infection and/or viral infection.

Neuropathy and myopathy share a common feature in that damage to the nerves and muscles, respectively, leads to an inflammatory response. In certain circumstances, damage to the nerves and muscles, respectively, may be caused by an autoimmune reaction to a self-antigen. Compositions and methods of the disclosure may be used to treat neuropathy, at least in part, because the compositions and methods of the disclosure reduce autoimmune and inflammatory responses.

### Common Cold

The common cold comprises a viral infectious disease of the upper respiratory tract which affects a subject's nose, sinus and throat. There are over 200 different viral strains that cause the common cold and each strain has a distinct set of symptoms. Usually a person will recover in about 1-2 weeks with the exception that a small number of the population will need 3-4 weeks before the symptoms abate. Infants and preschool children are susceptible to colds because they have not developed an immune system to many viruses. Older children and adults develop immunity to many of the viruses that cause the common cold. However, one can still have a cold when infected with a cold virus, because of a weakened immune system (through, for example, disease, immunosuppressive therapy, or age (either very young or old)).

Compositions of the disclosure may be used to treat the common cold. In particular, those compositions of the disclosure comprising a statin, an Omega-3 fatty acids or polyunsaturated oils and vitamin C may be used to treat the common cold. Patient A (see Examples) contracted the common cold. In this study the cold was gone within 3 days of initiating treatment with a composition of the disclosure. As the cold may otherwise have lasted several weeks, treatment according to the compositions and methods of the disclosure demonstrate a significant improvement over conventional therapies.

### Dementia

Dementia (also known as senility) represents a broad range of brain diseases that cause short term mild cognitive impairment (MCI) and eventually, severely impair a subject's basic cognitive functions. A chromic decline in memory with at least one additional cognitive function signals the onset of dementia. In addition to memory, cognitive functions including, but not limited to, aphasia (communication), apartia (motor execution), agnosiax (recognition) and executive function (synthesis) may be affected.

Dementia may be classified as vascular dementia, Alzheimer's disease, progressive supranuclear palsy, corticobasal degeneration, frontotemporal dementia and Lewy body dementia. Compositions and methods of the disclosure preserve a subject's cognitive functions and prevent further deterioration of memory.

Patient A's long standing use of statins has had a dramatic negative effect on his ability to remember names and organize daily routines. Patient A had taken bacopa, an ayurveda herb used in India to improve mental alertness, comprehension, memory and recollection. At the time, prior to his heart attack and after his quadruple bypass, bacopa had no effect on memory or recollection; however, after Patient A took Omega-3 fish oil and statin(s) the effect of bacopa was amplified. He had no problem with memory loss or organizing daily routines.

In combination with Omega-3 fish oil and a statin, Patient A was administered vinpocetine. Vinpocetine is an extract of the periwinkle plant that is an effective vasodilator and a nootropic for improving memory and cerebral metabolism.

Patient B started to have seizures in October, 2004 and continued to have minor seizures until September 2010 when the seizure intensity and duration began to increase. Consequently, her anti-seizure medication was increased to its maximum dosage. In January, 2015 she experienced a few auras or preictal events that immediately proceed the onset of a seizure. Until April 2015, the preictal events occurred every other day. In May 2015, she was taken off Crestor as a possible cause of the preictals, but the events still occurred. In June 2015, she was given vinpocetine. Since taking vinpocetine, the auras ceased and have not returned.

Kindling models in rats as well as Patient B's preictals demonstrate that vinpocetine exhibits anticonvulsant properties. Furthermore, vinpocetine may inhibit the upregulation of NF-kB by TNF2, thereby preventing inflammation.

Compositions of the disclosure, and, in particular, those comprising vinpocetine also significantly improved memory retention and muscular coordination in Patient A.

In addition to the diseases and disorders described above, compositions and methods of the disclosure may be used to treat any one or more of the following diseases and conditions.

TABLE 1
Diseases and Disorders
Acute disseminated encephalomyelitis (ADEM)
Addison's disease
Agammaglobulinemia
Alopecia areata
Amyotrophic lateral sclerosis (Also Lou Gehrig's disease; Motor Neuron Disease)
Ankylosing Spondylitis
Antiphospholipid syndrome
Antisynthetase syndrome
Atopic allergy
Atopic dermatitis
Autoimmune aplastic anemia
Autoimmune cardiomyopathy
Autoimmune enteropathy
Autoimmune hemolytic anemia
Autoimmune hepatitis
Autoimmune inner ear disease
Autoimmune lymphoproliferative syndrome
Autoimmune peripheral neuropathy
Autoimmune pancreatitis
Autoimmune polyendocrine syndrome
Autoimmune progesterone dermatitis
Autoimmune thrombocytopenic purpura
Autoimmune urticaria
Autoimmune uveitis
Balo disease/Balo concentric sclerosis
Behçet's disease
Berger's disease
Bickerstaffs encephalitis
Blau syndrome
Bullous pemghigoid
Cancer
Castleman's disease
Celiac disease
Chagas disease
Chronic inflammatory demyelinating polyneuropathy
Chronic recurrent multifocal osteomyelitis
Chronic obstructive pulmonary disease
Churg-Strauss syndrome
Cicatricial pemphigoid
Cogan syndrome
Cold agglutinin disease
Complement component 2 deficiency
Contact dermatitis
Cranial arteritis
CREST syndrome
Crohn's disease
Cushing's Syndrome
Cutaneous leukocytoclastic angiitis
Degos disease
Dercum's disease
Dermatitis heretiformis
Dermatomyositis
Diabetes mellitus type I
Diffuse cutaneous systemic sclerosis
Dressler's syndrome
Drug-induced lupus
Discoid lupus erythematosus
Eczema
Endometriosis
Enthesitis-related arthritis
Eosinophilic fasciitis
Eosinophilic gastroenteritis
Eosinophilic pneumonia
Epidermolysis bullosa acquisita
Erythema nodosum
Erythroblastosis fetalis
Essential mixed cryoglobulinemia
Evan's syndrome
Fibrodysplasia ossificans progressive
Fibrosing alveolitis (or Idiopathic pulmonary fibrosis)
Gastritis
Gastrointestinal pemphigoid
Glomerulonephritis
Goodpasture's syndrome
Granulomatosis with polyangiitis
Graves' disease
Guillain-Barré syndrome (GBS)
Hashimoto's encephalopathy
Hashimoto's thyroiditis
Henoch-Schonlein purpura
Herpes gestationis aka Gestational Pemphigoid
Hidradenitis suppurative
Hughes-Stovin syndrome
Hypogammaglobulinemia
Idiopathic inflammatory demyelinating diseases
Idiopathic pulmonary fibrosis
Idiopathic thrombocytopenic purpura (see Autoimmune thrombocytopenic purpura)
IgA nephropathy
Inclusion body myositis
Interstitial cystitis
Juvenile idiopathic arthritis aka Juvenile rheumatoid arthritis
Kawasaki's disease
Lambert-Eaton myasthenic syndrome
Leukocytoclastic vasculitis
Lichen planus
Lichen sclerosis
Linear IgA disease (LAD)
Lupoid hepatitis aka autoimmune hepatitis
Lupus erythematosus
Majeed syndrome
Ménière's disease
Microscopic polyangiitis
Miller-Fisher syndrome (see Guillain-Barre Syndrome)
Mixed connective tissue disease
Morphea
Mucha-Habermann disease aka Pityriasis lichenoides et varioliformis acuta
Multiple sclerosis
Microscopic colitis
Myasthenia gravis
Myositis
Narcolepsy
Neuromyelitis optica (also Devic's disease)
Neuromyotonia
Ocular cicatricial pemphigoid
Opsoclonus myoclonus syndrome
Ord's thyroiditis
Palindromic rheumatism
PANDAS (pediatric autoimmune neuropsychiatric disorders associated with streptococcus)
Paraneoplastic cerebellar degeneration
Paroxysmal nocturnal hemoglobinuria (PNH)
Parry Romberg syndrome
Parsonage-Turner syndrome
Pars planitis
Pemphigus vulgaris
Pernicious anaemia
Perienus encephalomyelitis
POEMS syndrome
Polyareteritis nodosa
Polymyalgia rheumatica
Polymyositis
Primary sclerosing cholangitis
Primary biliary cirrhosis
Progressive inflammatory neuropathy
Psoriasis
Psoriatic arthritis
Pyoderma gangrenosum
Pure red cell aplasia
Rasmussen's encephalitis
Raynaud phenomenon
Relapsing ploychondritis
Reiter's syndrome
Restless leg syndrome
Retroperitoneal fibrosis
Rheumatoid arthritis
Rheumatic fever
Sarcoidosis
Schizophrenia
Schmidt syndrome another form of APS
Schnitzler syndrome
Scleritis
Scleroderma
Serum Sickness
Sjögren's syndrome
Spondyloarthropathy
Still's disease see Juvenile Rheumatoid Arthritis
Stiff person syndrome
Subacute bacterial endocarditis (SBE)
Susac's syndrome
Sweet's syndrome
Sydenham chorea (see PANDAS)
Sympathetic ophthalmia
Systemic lupus erythematosus (see Lupus erythematosus)
Takayasu's arteritis
Temporal arteritis (also known as “giant cell arteritis”)
Thrombocytopenia
Tolosa-Hunt syndrome
Transverse myelitis
Ulcerative colitis (one of two types of idiopathic inflammatory bowel disease “IBD”)
Undifferentiated connective tissue disease different from Mixed connective tissue disease
Undifferentiated spondyloarthropathy
Urticarial vasculitis
Vasculitis
Vitiligo

### Definitions

Omega fatty acids and Omega oils: Omega-3 fatty acids include natural, manufactured or pharmaceutical fatty acids and fatty acid esters as well as other derivatives such as natural oils, congregates or salts. The fatty acid, fatty acid esters or fatty acid oils or ethyl-fatty acids or fatty acid alcohols or fatty acid glycols or fatty acid triglycerides that include, but are not limited to: Omega-3 polyunsaturated short-chain fatty acids with an aliphatic tail less than 6 carbon atoms; polyunsaturated medium-chain fatty acids with an aliphatic tail of 6-12 carbon atoms; polyunsaturated long-chain fatty acids with an aliphatic tail of 13-21 carbon atoms and very-long chain fatty acids with an aliphatic tail greater than 22 carbon atoms. The short chain and very-long chain fatty acids or esters include: myristoleic, palmitoleic, sapienic, oleic, elaidic, vaccenic, linoleic, linolelaidic, alpha-linolenic, arachidonic, eicosapentaenoic (EPA), alpha-linolenic, erucic and docosahexaenoic (DHA) acids. Also included are the saturated fatty acids that include: caprylic, capric, lauric, myristic, palmitic, stearic, arachidic, lignoceric and cerotic acid. The Omega-3 fatty acids with a statin(s) have a profound effect on the autoimmune diseases. Likewise Omega-6 fatty acids and Omega-9 fatty acids with a statin(s) have a similar effect on autoimmune diseases as well as the Omega-3 fatty acids. The Omega-6 fatty acids include arachidonic acid and alpha-linoleic acid. The Omega-9 fatty acids include oleic, erucic, elaidic, mead and nervonic acid.

Omega-3, Omega-6 and Omega-9 are effective in treating autoimmune diseases when administered with a statin. The ratio of different fatty acids is usually at a minimum of 1:1. That is the ratio of EPA to DHA is preferably 1:1; however, the ratio could be equally a 1:0 or 0:1 or 2:1. Most over the counter Omega-3 fatty acids are 2:1; however, other Omega-3 fatty acids are 1:2, EPA to DHA. For example, psoriasis required in Patient A an EPA of 180 mg and a DHA of 120 mg with a fish oil concentration of 600 mg and a statin(s). However, to effectively treat the various forms of keratosis, Patient A needed 1000 mg of Omega-3 fatty acids that included 667 mg of EPA and 333 mg of DHA. That was the minimum for Patient A. Increasing the concentration of fatty acids or fatty oils or fatty esters or fatty triglycerides or fatty acid alcohols did not relieve the symptoms of the disease, because the disease was treated at the lower concentration. The higher concentration just lowered the total cholesterol. The total cholesterol's homeostasis was established when an Omega-3 or Omega-6, or Omega-9 was combined with a statin(s). The omega fatty acids alone did not effectively treat the diseases rather it was the combination of omega fatty acids or mega oils and statin(s) that effectively treated the diseases.

Cholesterol lowering medicaments: Statins are also called HMG-CoA reductase inhibitors. They are drugs used to lower cholesterol levels by inhibiting the enzyme HMG-CoA reductase that is responsible for the preparation of cholesterol in the liver. The statins used herein as well as others include Atorvastatin (Lipitor or Torvast), Fluvastatin (Lescol), Lovastatin (Mevacor, Altocor, or Altoprev), Pitavastatin (Livalo or Pitava), Pravastatin (Provachol, Selektine, or Lipostat), Rosuvastatin (Crestor) and Simvastatin (Zocor or Lipex). There are several combinations of statins that are extremely effective, one such combination includes Ezetimibe/Simvastatin (Vytorin) or combinations of statins and vitamins such as Lovastin/Niacin (Advicor), Simvastatin/Niacin (Simcor) or combinations of 2 different functional drugs such a Atorvastatin/Amlodipine hesylate (Caduet). There are other cholesterol lowering drugs that act by decreasing cholesterol in the small intestine such a drug is Ezetimibe (Zetia or Ezetrol). Any drug that lowers cholesterol is a prime candidate for treating diseases with another medicament. The cholesterol lowering drug(s) or medicament(s) cannot effectively treat diseases as a monotherapy; they need another medicament to aid in treating the disease.

Other cholesterol lowering medicaments include fibrates, amphipathic carboxylic acids, such as Bezafibrate (Bezalip), Ciprofibrate (Modalim), Gemfbroizil (Lopid), Fenofibrate (Tri Cor) or Clinofibrate (Lipoclin). Other cholesterol lowering medications as well as vitamins include niacin and bile acid sequestrants such as Colestipol (Colestid), Cholestyramine (Questron), Endur-Acin and Colesevelam (Welchol).

There is another cholesterol lowering medicament that inhibits synthesis of an enzyme PCSK9 that binds to the receptors of low density lipoprotein (LDL) cholesterol resulting in making the LDL receptor more effective in removing LDL from the blood. The new drugs are Alirocumab and Evolocumab. Any cholesterol lowering medicament in combination with a fatty acid(s), fatty acid ester(s), fatty acid triglycerides(s) or the like will treat diseases. It is proven herein that the concentration of cholesterol lowering medicament(s) or fatty acid(s) is not significant and a minimum amount is only needed. Also the type of cholesterol lowering medicament or fatty acid is not significant because the level of total cholesterol homeostasis will be at that level necessary to treat the disease.

Vitamins: Vitamins are essential organic compounds that an organism requires for survival, but cannot manufacture the organic compound in sufficient quantities; as is the case of vitamin D that requires sun exposure. There are 13 vitamins that are classified by their biological and chemical activity. Vitamins have many diverse biochemical functions. As shown herein the functions of the vitamins are not well understood.

For example, vitamin D may be classified as a hormone and functions as a regulator of mineral metabolism, treats rickets in children and osteoporosis in the elderly; but, herein, it has been shown to treat myopathy and neuropathic pain. With the new combination of fatty acid(s), cholesterol lowering medicament(s) and vitamin D the range of disease(s) treated is extended to additional and various inflammatory diseases.

Vitamin D is a secosteroid comprising vitamin D₃ (Cholecalciferol) and vitamin D₂ (Ergocalciferol). Either forms of vitamin D are beneficial in treating myopathy, myositis and neuropathy. The vitamin D used herein was vitamin D₃ and the minimum concentration was 2000 IU that resulted in a blood plasma vitamin D₃ 25-OH level greater than 30 ng/ml. However, at a vitamin D₃ intake of 12,000 IU, the total cholesterol level increased, but myopathy, myositis or neuropathy did not return; rather, the diseases were still abated.

Vitamin C like vitamin D is an essential in human vertebrates. There are 2 forms found in nature for vitamin C, ascorbate as L-ascorbate and ascorbic acid as L-ascorbic acid. Both are present in human vertebrates and are conformational isomers. Herein, vitamin C was used in the form of L-ascorbic acid. By itself vitamin C has a fluctuating effect in biological reactions. Treating a particular disease requires a certain concentration while treating another disease requires a different concentration and, likewise, treating a viral infection, the common cold, requires a very different concentration.

The connection between treating diseases and viral infections that specifically require the use of vitamin C, also require fatty acid(s) and a cholesterol lowering medicament. Controlling the total cholesterol is a cause and effect result of combining a fatty acid and a cholesterol lowering medicament. Adding vitamin C to the matrix adds to the enhanced treating of autoimmune diseases and viral infections. The spongy gum disease requires at least 1000 mg/day of vitamin C; whereas, the common cold, a viral disease, requires 8000 mg/day.

Organic Compounds: Bacopa is an aquatic plant that belongs to the family Plantaginaceae. It is an herb used in Indian culture as an ayurvedic medicine to treat anxiety and memory disorders. Bacopa monnieri was used exclusively herein; however, any kingdom of Plantal is contemplated. The minimum is 250 mg/day; however, for a stronger cognitive awakening, 500 mg/day is preferred.

Vinpocetine has been reported to have vasodilation and nootropic improvement in cognitive functions and cerebral metabolism. Also it has been reported as a dominant anti-inflammatory agent in the treatment of Parkinson's disease and Alzheimer's disease.

Vinpocetine in Patient B at 30 mg/day inhibited preictals in a patient accustomed to seizures. In Patient A, the bacopa expanded memory and learning capabilities and the vinpocetine improved the cognitive functions as well as cerebral metabolism. All of this was accomplished with the addition of concurrent administration of fatty acid(s) and a cholesterol lowering medicament(s). As a semi synthetic derivative alkaloid of vincamine or any other alkaloid derivative including, but is not limited to, reserpine, reserpinine, akuammicine, majdine, vinerine, ervine, vineridine, tombozine, vincananine, vincanidine, vincamone, apovincamine, vincaminol and perivincine are examples of cognitive enhancing medicaments.

Excipients: Excipients are substances used along with the active ingredient of a medication. Their purposes are many from such important enhancements as absorption and solubility of the active ingredient to viscosity reduction, to shelf life of the overall medication and to enhancement of manufacturing processes. The types of excipients include, but are not limited to: anti-adherents, binders, coatings, color, disintegrants, encapsulants, flavors, glidants, lubricants, preservatives, sorbents and sweeteners. Compositions and methods of the disclosure may be administered as a solid tablet or gel capsule or injection or vaporizer.

The term “total cholesterol percent relative range” is meant to describe:

[(the maximum total cholesterol level−the minimum total cholesterol level)/((the maximum total cholesterol level+the minimum total cholesterol level)/2)]×100.

The term “total cholesterol relative range” is meant to describe:

(the maximum total cholesterol level−the minimum total cholesterol level)/((the maximum total cholesterol level+the minimum total cholesterol level)/2).

At least two data points are collected to determine a total cholesterol value. A first data point may be collected prior to administration of a composition of the disclosure and a second data point may be collected after administration of the composition of the disclosure. Alternatively, or in addition, a third or subsequent data point may be collected following a treatment change in the components of the composition, one or more dosages/dosing schedules of the components of the composition or a change of formulation of one or more components of the composition. As treatment with a composition of the disclosure may decrease the subject's cholesterol level between measurements, the first or earlier data point may represent the maximum total cholesterol and the second, third, subsequent or latest data point may represent the minimum total cholesterol. Alternatively, should treatment with a composition of the disclosure may increase the subject's cholesterol level between measurements, the first or earlier data point may represent the minimum total cholesterol and the second, third, subsequent or latest data point may represent the maximum total cholesterol.

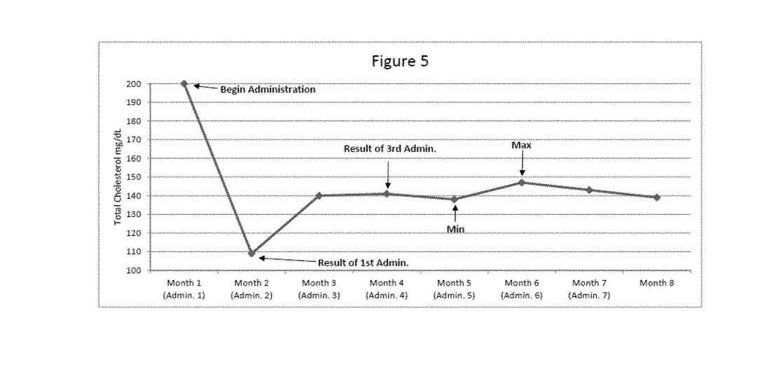

In certain embodiments, a first data point may be collected after daily administration of a composition of the disclosure for at least three months and additional (second, third, and subsequent) data points may be collected thereafter weekly or monthly for a period of at least 2, 3, 4, 5, 6, 7, 8, 9, 10, 11 or 12 months. FIG. 5 provides an exemplary timeline illustrating these embodiments. From collected and analyzed samples, a maximum total cholesterol (or highest cholesterol measurement) is determined and from the same sample, a minimum total cholesterol value (or lowest cholesterol measurement) is determined.

In certain embodiments, the at least two data points should be measured/collected monthly for a period of at least 2, 3, 4, 5, 6, 7, 8, 9, 10, 11 or 12 months. In certain embodiments, the at least two data points should be measured/collected two months apart. In certain embodiments, the last of the at least two data points should be measured/collected after all changes to the medication have been made during, for example, a treatment schedule or clinical trial. In the context of a clinical trial, individual subject data may be kept separate or may be pooled prior to data analysis.

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Embodiments defined by each of these transition terms are within the scope of this invention.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The terms “treating” and “treatment” as used herein refer to reduction in severity and/or frequency of symptoms, elimination of symptoms and/or underlying cause, and improvement or remediation of damage. The phrase “administering to a subject” refers to the process of introducing a composition or dosage form of the invention into the subject (e.g., a human or other mammalian subject) via an art-recognized means of introduction.

By the terms “effective amount” and “therapeutically effective amount” of an agent, compound, drug, composition or combination of the invention which is nontoxic and effective for producing some desired therapeutic effect upon administration to a subject or patient (e.g., a human subject or patient).

### Pharmaceutical Formulations

Compositions of the disclosure comprising at least one fatty acid and at least one cholesterol lowering compound may be provided in any formulation or any route of administration. In certain embodiments, compositions of the disclosure are formulated for oral administration as, for example, a liquid, a tablet, a capsule, a caplet, or a particulate (either in a liquid suspension, a solid dosage form, or an encapsulated form).

Nonlimiting examples of formulations are provided below.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like.

Capsules are also preferred oral dosage forms, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited earlier herein, which describes materials and methods for preparing encapsulated pharmaceuticals.

Oral dosage forms, whether tablets, capsules, caplets, or particulates, can, if desired, be formulated so as to provide for controlled release and/or sustained release, i.e., gradual, release of a composition of the disclosure, from the dosage form to the patient's body over an extended time period, typically providing for a substantially constant blood level of the at least one fatty acid or the at least one cholesterol lowering compound over a specific time period (e.g. 1, 2, 3, 4, 5, 6, 7, 8, 9, 10, 11, 12, 16, 24, 36, or 72 hours or any time period in between). Release of the composition may also be delayed; that is, there is a time lag between administration and the start of release of the composition.

Generally, as will be appreciated by those of ordinary skill in the art, sustained release dosage forms can be formulated by dispersing the active agent within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone e.g., Povidone K30, polyvinyl acetate, and ethylene-vinyl acetate copolymer. Preferred sustained release polymers herein include those available as “Methocel” polymers from Dow Chemical, particularly the methylcellulose ether polymers in the Methocel™ A group, having a viscosity grade of about 4,000 cps and a methoxyl content of about 27.5% to 31.5%, e.g., Methocel™ A 15LV, Methocel™ A15C, and Methocel™ A4M.

When sustained release preparations are prepared, tablets, granules, powder, capsules, and the like can be produced according to a conventional method after adding excipient, and as necessary, binder, disintegrating agent, lubricant, coloring agent, taste-modifying agent, flavoring agent, and the like. These additives may be ones generally used in the field, and for example, lactose, sodium chloride, glucose, starch, microcrystalline cellulose, and silicic acid as the excipient, water, ethanol, propanol, simple syrup, gelatin solution, hydroxypropyl cellulose, methyl cellulose, ethyl cellulose, shellac, calcium phosphate, and polyvinylpyrrolidone as the binder, agar powder, sodium hydrogen carbonate, sodium lauryl sulfate, and stearic acid monoglyceride as the disintegrating agent, purified talc, stearic acid salt, borax, and polyethylene glycol as the lubricant, (3-carotene, yellow iron sesquioxide, and caramel as the coloring agent, and saccharose and orange peel as the taste-modifying agent can be listed as examples. It should be noted that various grades of microcrystalline cellulose are preferred fillers herein, e.g., Avicel® PH1O1, Avicel® PH 102, and Avicel® PH200 (FMC), with particle sizes of about 50 microns, 100 microns, and 190 microns, respectively. Microcrystalline cellulose having a particle size in the range of about 50 microns to 200 microns is preferred herein.

The dosage forms may also be provided with a delayed release coating, e.g., composed of an acrylate and/or methacrylate copolymers. Examples of such polymers are those available under the trade name “Eudragit” from Rohm Pharma (Germany). The Eudragit series E, L, S, RL, RS, and NE copolymers are available as solubilized in organic solvent, in an aqueous dispersion, or as a dry powder. Preferred acrylate polymers are copolymers of methacrylic acid and methyl methacrylate, such as the Eudragit L and Eudragit S series polymers. Other preferred Eudragit polymers are cationic, such as the Eudragit E, RS, and RL series polymers. Eudragit ElOO and E PO are cationic copolymers of dimethylaminoethyl methacrylate and neutral methacrylates (e.g., methyl methacrylate), while Eudragit RS and Eudragit RL polymers are analogous polymers, composed of neutral methacrylic acid esters and a small proportion of trimethylammonioethyl methacrylate.

Preparations according to this invention for parenteral administration include sterile aqueous and nonaqueous solutions, suspensions, and emulsions. Injectable aqueous solutions contain the active agent in water-soluble form. Examples of nonaqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

Compositions of the disclosure may also be administered through the skin using conventional transdermal drug delivery systems, wherein the active agent is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. Transdermal drug delivery systems may in addition contain a skin permeation enhancer.

In addition to the formulations described previously, the composition may be formulated as a depot preparation for controlled release of the at least one fatty acid or the at least one cholesterol lowering compound, preferably sustained release over an extended time period. These sustained release dosage forms are generally administered by implantation (e.g., subcutaneously or intramuscularly or by intramuscular injection).

Although the present compositions will generally be administered orally, parenterally, transdermally, or via an implanted depot, other modes of administration are suitable as well. For example, administration may be transmucosal, e.g., rectal or vaginal, preferably using a suppository that contains, in addition to the active agent, excipients such as a suppository wax. Formulations for nasal or sublingual administration are also prepared with standard excipients well known in the art. The pharmaceutical compositions of the invention may also be formulated for inhalation, e.g., as a solution in saline, as a dry powder, or as an aerosol.

The term “dosage form” denotes any form of a pharmaceutical composition that contains an amount of active agent sufficient to achieve a therapeutic effect with a single administration. When the formulation is a tablet or capsule, the dosage form is usually at least one such tablet or capsule. The frequency of administration that will provide the most effective results in an efficient manner without overdosing will vary with the characteristics of the particular active agent, including both its pharmacological characteristics and its physical characteristics, such as hydrophilicity.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree.

As used herein, “subject” or “individual” or “patient” refers to any subject for whom or which therapy is desired, and generally refers to the recipient of the therapy to be practiced according to the invention. The subject can be any vertebrate, but will typically be a mammal. If a mammal, the subject will in many embodiments be a human, but may also be a domestic livestock, laboratory subject or pet animal.

All percentages and ratios are calculated by weight unless otherwise indicated.

All percentages and ratios are calculated based on the total composition unless otherwise indicated.

Every maximum numerical limitation given throughout this disclosure includes every lower numerical limitation, as if such lower numerical limitations were expressly written herein. Every minimum numerical limitation given throughout this disclosure will include every higher numerical limitation, as if such higher numerical limitations were expressly written herein. Every numerical range given throughout this disclosure will include every narrower numerical range that falls within such broader numerical range, as if such narrower numerical ranges were all expressly written herein.

The values disclosed herein are not to be understood as being strictly limited to the exact numerical values recited. Instead, unless otherwise specified, each such value is intended to mean both the recited value and a functionally equivalent range surrounding that value. For example, a value disclosed as “20 mg/dL” is intended to mean “about 20 mg/dL.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the disclosure have been illustrated and described, various other changes and modifications can be made without departing from the spirit and scope of the disclosure. The scope of the appended claims includes all such changes and modifications that are within the scope of this disclosure.

### EXAMPLES

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### Example 1: Patient Demographics

The data presented herein were collected over a 14 year period from a 69 year old, Caucasian male (Patient A) and a 72 year old Caucasian female (Patient B).

Patient A had several heart attacks in 1991, 1995 and 2001. The heart attack in 1991 resulted in administering a balloon angioplasty. The attack in 1995 resulted in the placement of a stent in the coronary artery. In 2001, the heart attack resulted in a quadruple bypass. After years without a heart incident, Patient A suffered arterial fibrillation in March 2010. Patient A was implanted with a defibrillator in the upper left chest along with cardiac ablation.

There were a number of statins prescribed for Patient A beginning in 2001 with Lipitor, Vytorin, Provachol and Zetia. Statins were changed continually because the insurance provider no longer listed a particular statin on its formulary. The only non-statin was Tricor, a fenofibrate, which reduces cholesterol and triglycerides.

After suffering arterial fibrillation, Patient A was prescribed a series of medicaments to control the fibrillation, blood pressure and water retention. The prescribed medications included Lisinopril, Digoxin, Warfarin, Metroprolo and Lasix.

The medicament Metroprolol alone is known to cause psoriasis and a combination of Metoprolol and statin are known to cause psoriasis and other autoimmune diseases.

A 72 year old female Caucasian, Patient B, does not have a heart disease or any autoimmune disease; however, she has had seizures beginning in 2004. Magnetic resonance imaging (MRI) showed a small scar in the hippocampus region and is believed to cause her seizures. The seizures are controlled by two drugs, Depakote and Keppra.

The reason to include Patient B in this study is because Patient B and Patient A are spouses and work and eat meals together. Whatever Patient A consumes so does Patient B. The vitamins, hormones, fatty acids and minerals are taken by both Patients under the same conditions. Patient B becomes a baseline (negative control) to measure the effects of vitamins, hormones, fatty acids, organic compounds and minerals taken without statins.

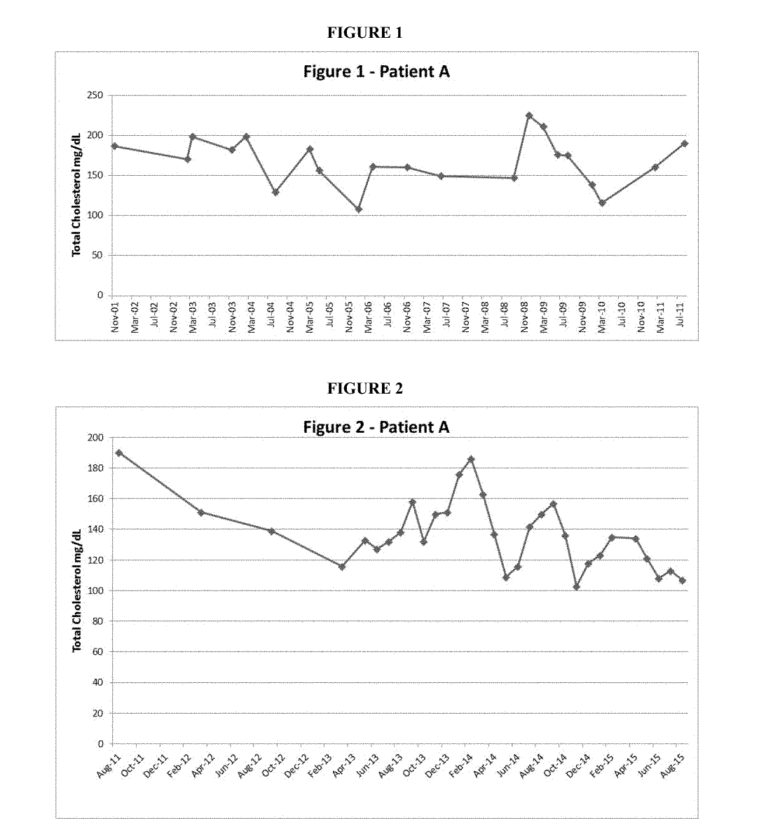

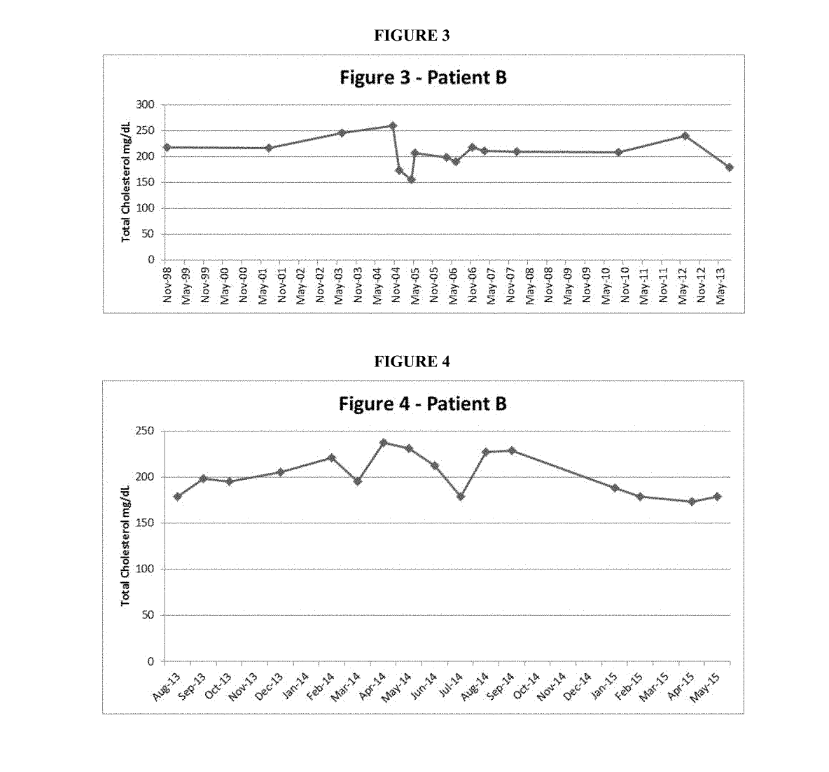

The effect of taking a fatty acid, hormone, vitamin or organic compound while taking a statin or other cholesterol lowering medicine(s) was measured in Patient A using Patient B as a negative control. Both Patients A and B took the same fatty acids, hormones, vitamins, minerals, organic compounds and antioxidants. Also they had the same meals each day, 7 days a week. A comparison of FIG. 1 (Patient A) and FIG. 3 (Patient B) reveals striking results provided herein.

The concentration of medicaments will vary depending on numerous factors, such as, for example, the seriousness of the disease; use of other medications; the subjects physiology; other comorbidities; medicament preparation, for example, pill versus gel capsule; medicament administration and the type of disease being treated.

### Example 2: Experiment 1

Patient B's medical history began on July, 2001. Her total cholesterol was 217 mg/dL. The total cholesterol increased to 259 mg/dL in October, 2004. But in December, 2004, the total cholesterol decreased to 174 mg/dL, a total decrease of 32.8%. At the time, Patient B was under the care and guidance of a naturopathic doctor who advised Patient B to undergo a bodily detoxification. The detoxification process is described in Table 6. The goal was to take for 2 weeks: greens and acidophilus (2 times daily), Ultra Clear or Plus (3 times daily) and Meta fiber or flaxseed (2 times daily). The purpose was to rid the body's cells of waste and toxins. What resulted was a lowering of the total cholesterol by 32.8%, a very impressive decrease. Patient B was given 3 months to recuperate from the detoxification before she went through the detoxification program again for another 2 weeks.

In April, 2005, Patient B's total cholesterol level was measured. From October, 2004 her total cholesterol value decreased by 38%. That is from the December, 2004, her total cholesterol decreased by another 5.2%. This is a substantial decrease in total cholesterol, lipoproteins and hormones just by taking a few greens, acidophilus, minerals, vitamins, flax seed and fiber.

Once the detoxification was over, the blood's total cholesterol, lipoproteins and hormones, returned to a normal value. This oscillatory cholesterol value lasted from May, 2005 through June, 2012, more than 7 years as illustrated in FIG. 3 and FIG. 4.

### Example 3: Experiment 2

Table 3 has 2 interesting data points that will be used later in establishing the meaning of vitamin D. Those data points are September, 2010 and June, 2012. The total cholesterol increases and the blood plasma concentration of total vitamin D increases. From 2 data points and the high blood plasma concentration of vitamin D, one can conclude that very high concentrations of vitamin D in blood serum will increase total cholesterol.

For the next year, August, 2013, the cholesterol level dropped to 179 mg/dL when Patient B took fish oil, Omega-3 fatty acid ethyl ester with a concentration of 2100 mg of fish oil, containing EPA-800 mg and DHA-400 mg. In the mixture taken on August, 2013 were the ingredients of vitamin C, vitamin E, calcium, magnesium and vitamin D.

As shown in Table 3, the lower cholesterol is due to the Omega-3 fatty acids and low concentration of vitamin D. From August, 2013, FIG. 4, through September, 2014, the total cholesterol constantly oscillates from a maximum of 237 mg/dL to a minimum of 179 mg/dL a total change of 32.4%. Such a large fluctuation is not a homeostatic control limit.

### Example 4: Experiment 3

From August, 2013 through September, 2014, there were no signs of treating diseases, autoimmune or otherwise. Patient B was given Crestor, (20 mg) resulting in a significant decrease in cholesterol. The total cholesterol was homeostatic at a maximum of 179 mg/dL and a minimum of 173 mg/dL for a percentage relative range of 3.4%, a very tight range for total cholesterol.

### Example 5: Experiment 4

Experiment 4 is a subset of Experiment 3. Patient B was given Crestor (20 mg) and her cholesterol level was measured from January, 2015 through April, 2015. During the experiment, she experienced auras (preictals), brief moments where she was unable to form a vocal sentence. It is usual that her preictals occur before a grand mal seizure. At first her auras occurred monthly. In April they increased to every other day without a grand mal seizure. In May, she discontinued Crestor, but the auras continued with the same frequency. The following month, June, she was given vinpocetine and Coenzyme Q10 (trans-form). The auras discontinued the next day.

In summary from Patient B's data illustrated in FIG. 3-4 and Table 3, we conclude that: (1) total cholesterol oscillates continuously over a broad range; (2) a drastic change in diet, for example, detoxification for one week, drastically reduces total cholesterol and subdues total cholesterol for a number of years, in this case approximately 7 years; (3) an addition of Omega-3 fatty acids initially shocks the human system similar to detoxification, but continual administration of the fatty acids (triglycerides or ethyl ester fatty acids) or hormones or vitamins does not stabilize the total cholesterol; (4) an addition of a statin to the Omega-3 fish oil and hormone or vitamin, now called a medicament, stabilizes the total cholesterol within very narrow limits.

### Example: Experiment 5

Patient A sheds much of the insight into treating autoimmune diseases and other diseases. The data was collected over a 14 year period. Experiment 5 begins in January, 2001 and ends November, 2009. The statins administered during this time were modified, added, changed, substituted, eliminated and adjusted according to health insurance formularies. The additions and modifications were graphically represented in FIG. 1-2 and illustrated in Table 2.

Patient A underwent a quadruple bypass in May, 2001. He was prescribed Lipitor and Lopressor shown in Table 2. That changed in March, 2003 to Lipitor and Metoprolol. In an attempt to lower Patient's A total cholesterol, the Lipitor concentration was increased from 40 mg/day to 80 mg/day on February, 2004.

The blood work done in August, 2004 gave a total cholesterol of 129 mg/dL a 69% decrease from the present February reading of 198 mg/dL; however, continuing with the same statin concentration did nothing to maintain the same or similar total cholesterol level. In fact the total cholesterol increased drastically to 183 mg/dL. Increased levels of statins give a drastic decrease in total cholesterol levels, but the change is not permanent and conditions revert to their previous state.

In May, 2005, the medication was enhanced by the inclusion of Tricor. The total cholesterol decreased to 156 mg/dL.

In January, 2006, Patient A started a 2 week detoxification program as illustrated in Table 6 (the same program started by Patient B in December 2004 and April 2005). Results for Patient A and Patient B were similar; a drastic reduction in total cholesterol. For Patient A whose total cholesterol was 187 mg/dL, ingesting Lipitor at 40 mg per day, the detoxification lowered the total cholesterol to 108 mg/dL, a change of −42.2%. Total cholesterol decreased by 42% just by detoxifying the body.

Without taking any statins, Patient B had a similar reaction to the detoxification. The purpose of discussing detoxification is the drastic effect it has on overall hormone synthesis. Whether or not detoxification is useful in treating autoimmune diseases; its effect on the human body is what is important. The same effect was observed with respect to Patient A and Patient's B's consumption of Omega-3 fatty acids. Total cholesterol will drastically decrease with the consumption of an Omega-3 fatty acid(s) or oil(s) and will decrease with the consumption of Omega-3 fatty acid(s) and a statin(s), but in the case of an Omega-3 fatty oil(s) and a statin(s) the result is to lower the total cholesterol and maintain that level for a considerable time within a narrow tolerance range establishing total cholesterol homeostasis.

The combination of an oil and a statin results in homeostasis. Another consequence of combining Omega-3 fatty acid(s) or oil(s) and a statin(s) are to treat autoimmune diseases. As will be discussed, other vitamin(s), hormone(s), organic compound(s), enzyme(s), protein(s) and antibodies will treat other autoimmune diseases or other diseases and in the process establish a total cholesterol homeostasis.

Also, total cholesterol (TC) will affect high density lipoproteins (HDL), low density lipoproteins (LDL), triglycerides (TG) through the Friedwald equation TC=HDL+LDL+(0.2TG). Controlling the total cholesterol regulates very low density lipoproteins (VLDL), apolipoprotein A1, apolipoprotein B, an intermediate density lipoprotein (IDL), apolipoproteins, chylomicrons, enzymes, vitamins, hormones, fats, proteins, lipids, glycerol, etc.

After detoxification, Lipitor and Tricor administration returned the total cholesterol to the previous level of May, 2005.

In November, 2006 Patient A was put on Vytorin. The result stabilized total cholesterol. Again, as previous, Patient A was taken off Vytorin due to insurance formularies and he was put on Provachol (80 mg) and Zetia (10 mg). Total cholesterol was reduced slightly.

In August, 2009, Tricor was added to Lipitor in an attempt to reduce total cholesterol and triglycerides more specifically to reduce LDL and VLDL as well as increase HDL. The Lipitor and Tricor combination was a dismal failure.

A new combination was tried in January, 2010 with excellent results. Total cholesterol dropped to 138 mg/dL from its 175 mg/dL a total change of −21.1%. In March, 2010 the total cholesterol dropped further to 116 mg/dL. Again comparing August data to March data, there was a total change of −33.7%. The result showed a drastic lowering of the total cholesterol.

### Example 7: Experiment 6

April, 2010, Patient A's heart went into arterial fibrillation. It was decided to implant a defibrillator in the left chest.

Thereafter Patient A was prescribed a number of medications in addition to Lipitor and Zetia. The additional medications included Lisinopril, Digoxin, Warfarin, Metoprolol and later Lasix. Table 4 lists the medication and their concentrations in milligrams per day (mg/day).

After the implant, taking Lipitor and Zetia did not perform as stellar as in January, 2010. The total cholesterol values oscillated from a high in August, 2011 of 190 mg/dL to a low in September, 2012 to 139 mg/dL a percentage relative range of 164.5%. This value was not within the range of homeostasis.

Patient A was prescribed a number of medications. One of which was Metoprolol. As an adverse side effect, Metoprolol is known to cause psoriasis.

Ten months after taking Metoprolol, Patient A suffered from psoriasis on the left and right calves. By August, 2011 the psoriasis included the calves and the entire back. Warfarin, taken daily as a blood thinner, caused the psoriasis on the calves to bleed excessively due to the dermal skin layer cracking and separating from the epidermal layer.

By March, 2012, the psoriasis had progressed to the scalp. The psoriasis measured 1-1.5 inches in diameter and as the epidermal layer was removed by scratching the scalp or vigorously rubbing the scalp would remove large areas of hair.

By September, 2012, the ears were next affected resulting in excessive skin loss. Each epidermal layer would peel off from the ear. Later in February, 2013, psoriasis appeared on the face, 3 dots as precursors to plaque psoriasis.

### Example 8: Experiment 7

In March, 2013, Patient A took Omega-3 fish oil, vitamin D₃, vitamin C and a vitamin B complex. The itching from psoriasis was gone in 2 weeks. There was no calf bleeding and no gum bleeding. During the same time, myositis disappeared. Within the next 2 weeks, one month in all, all psoriasis had disappeared. The total cholesterol in September, 2012 was 139 mg/dL.

September's cholesterol reading was a result of taking Lipitor (80 mg) and Zetia (10 mg). The same concentration taken back in January, 2010 through September, 2012 resulted in average total cholesterol of 149 mg/dL. Comparing the average total cholesterol data calculated above at 149 mg/dL with March, 2013 data of 116 mg/dL, one observes a total cholesterol change of −22.1%, a very substantial decrease.

Total cholesterol was lowered significantly on a number of occasions. They were the detoxification of Patient B and Patient A and when Patient A was taking a high concentration of a new statin or multiple statins; however, the total cholesterol reverted to its previous high values and continued thereafter to oscillate. But, from May, 2013 through July, 2013, the total cholesterol's range was 5.0 mg/dL and the percentage relative range was 3.9%, a very tight range. A similar observation was made in Experiment 3 with Patient B.

### Example 9: Experiment 8

It is important to understand that Patient A took the medicaments 30 days prior to his blood specimen collected and a comprehensive metabolic panel and lipid panel evaluated in March, 2013. Seven days after the medicaments were ingested, the itching on the dermal and epidermal skin surface ceased. Another 7 days and the psoriasis on the face disappeared.

Within 14 days thereafter, all the psoriasis on the calves, back, scalp and ears was gone; hence, a 30 day period is all that is necessary for Patient A to be evaluated for effective remedies attributed to a medicament. Blood work is evaluated approximately every 30 days after March, 2013.

After March, 2013, vitamin C was the first medicament to be removed. Taking vitamin C for 2 months was enough to stop the gum bleeding and strengthen the gum tissue. Removal of vitamin C consequently had no effect. Also, there was no effect on the total cholesterol.

The vitamin C was replaced and Omega-3 fish oil removed. Within 2 weeks in August, 2013, psoriasis returned as well as another skin disease known as keratosis, both pilaris and seborrheic. Patient A was given 2000 mg of Omega-3 fish oils as outlined in Table 2 and cross-referenced in Table 4. Within 2 weeks the psoriasis and pilaris keratosis were gone. The seborrheic keratosis took 4-6 months to disappear.

Throughout the time interval May, 2013 through October, 2013, the total cholesterol remained within a very narrow range even when the Omega-3 fish oils were removed for 2 weeks. The total cholesterol high at 138 mg/dL was not included in Experiment 7's percent relative range, because the Omega-3 fish oils have a strong influence on the total cholesterol. This will be evident in later Experiments.

There was one exception to Experiment 8. In September, 2013, the total cholesterol was 158 mg/dL. Table 2 illustrates that the only significant change was the vitamin D₃ dosage. It was 12,000 IU units.

From the previous months, the dosage of vitamin D₃ ranged from 3000 IU to 5000 IU and to 6000 IU with no apparent effect on the total cholesterol level. At a vitamin D₃ consumption of 5000 IU, the total vitamin D, 25-OH in the blood serum measured 44.6 ng/mg; however, at a very high concentration of vitamin D₃, 12,000 IU; total vitamin D, 25-OH measured was 89.3 ng/ml; the total cholesterol level increases, but there was no change in autoimmune disease characteristics or other disease characteristics.

Vitamin D₃ may lower active statin metabolites. Thus, increasing the vitamin D₃ concentration may lower the efficacy of Atorvastatin.

Patient A went back to his normal medicament concentrations in October, 2013 and returned to a total cholesterol value of 132 mg/dL.

In November and December, 2013, the vitamin D₃ was split into a vitamin D₃, 1000 IU power tablet and a vitamin D₃, 1000 IU gel capsule. The total cholesterol increased slightly to 150 mg/dL. Psoriasis and keratosis did not return. Myopathy was slight. On a scale of 0-10, 0 being no pain and 10 being severe pain, Patient A's pain was about 1. Patient A has had myositis since he started taking statins in June, 2001. The level of pain increased progressively over the years reaching a level of 4 in March, 2013. Thereafter the myositis disappeared.

The indication was that powder vitamin D₃ does not have an effect on total cholesterol nor an effect on myopathy; also it is indicated that more than 1000 IU of vitamin D₃ as a liquid gel is needed to control myopathy for Patient A.

### Example 10: Experiment 9

In January, 2014, Patient A was taken off Lipitor and given Simvastatin at 40 mg. Zetia (10 mg) and Simvastatin (40 mg) is known also as Vytorin. Simultaneously Patient A discontinued taking vitamin D3. The first night after taking the Zetia/Simvastatin combination minus the vitamin D3, he experienced severe myositis at a pain level of 8 out of 10. Pain persisted throughout the early morning. Exercise lowered the pain level to 2 throughout the day.

It was the Simvastatin that cause a high level of pain. The next day he was given another 40 mg of Simvastatin along with Zetia and without vitamin D. Patient A had another painful night. The pain level was again 8. Patient A was given vitamin D₃ 1000 IU in solid tablet and vitamin D₃ 1000 IU added to the Omega-3 fish oil. The pain persisted. The vitamin D₃ powder provided little to no benefit and the vitamin D₃ 1000 IU added to the Omega-3 fish oils was not enough. Total vitamin D, 25-OH was 35 ng/ml.

Due to the high pain level, much larger vitamin D₃ concentration was administered. Vitamin D₃ was increased to 3000 IU. Again the same statins were administered as before without any myositis (myopathy). For 3 months, Patient A had no psoriasis, no keratosis, no bleeding gums and no myopathy. However, because the statin combination of Zetia/Simvastatin is not as efficacious as the statin combination Zetia/Lipitor, the total cholesterol increased, but remained in a narrow range. The total cholesterol range was 23 mg/dL and the percent relative range was 13.1% that is higher than the Lipitor/Zetia combination.

There were two regions in FIG. 2 that illustrate the relationship between Omega-3 fish oil and statin(s). Region 1 begins May, 2013 through October, 2013. Region 2 begins January, 2014 through March, 2014. Region 1 was influenced by Lipitor/Zetia and Omega-3 fish oil. Region 2 was influenced by Simvastatin/Zetia and Omega-3 fish oil. Each region had a different total cholesterol range. Lipitor had a lower range than Simvastatin, but Lipitor was at twice the concentration as Simvastatin. At the same concentrations each statin would perform in a similar manner. Region 1 had average total cholesterol of 132 mg/dL.

Region 2's average total cholesterol was 175 mg/dL. Both regions' total cholesterol differed by 43 mg/dL, a considerable amount given that the total cholesterol is less than 200 mg/dL for a patient with coronary heart disease. The same analogy holds through later experiments with different statins and different Omega-3 fish oil(s).

Whatever the level of total cholesterol, Patient A had no recurrence of psoriasis, no recurrence of keratosis, no recurrence of bleeding gums, no recurrence of neuropathy and no recurrence of myositis. The level of total cholesterol was indifferent to the treating of an autoimmune disease. The Omega-3 fatty oils are responsible for the narrow total cholesterol range and responsible along with a statin(s) for treating autoimmune diseases and other diseases.

Other organic compounds will treat other diseases aside from autoimmune diseases. Also, other organic compounds will narrow and still lower again the total cholesterol. Vitamin D₃ was an active ingredient for treating myopathy that required the combination of Omega-3 fish oil(s) and statin(s) to attain a superior efficacy of treatment.

### Example 11: Experiment 10

In April, 2014, Patient A was administered Crestor (40 mg) as a replacement for Lipitor (80 mg). The difference in total cholesterol for either statin was very slight; however, the concentration of Crestor was half that of Lipitor. Likewise the autoimmune diseases were suppressed with Omega-3 fish oil and Crestor and Omega-3 fish oil, Crestor and vitamin D₃.

Patient A was administered Omega-3 fish oil(s) at a concentration of 3000 mg. The fish oil(s) EPA (eicosapentaenoic acid) and DHA (docosahexaenoic acid) were now 3 times what they were in previous months. The result was a drastic decrease in total cholesterol. The percentage change was −20%; it represented the total cholesterol for April, 2014 of 137 mg/dL to May, 2014 whose total cholesterol was 109 mg/dL. There was no change in the autoimmune diseases.

### Example 12: Experiment 11

Experiment 11 was designed to check the results found in Experiment 10. Patient A changed his Crestor (40 mg) to Lipitor (80 mg). The total cholesterol increased substantially, because Crestor is a more efficacious inhibitor of the enzyme HMG-CoA reductase. The Lipitor/Zetia statins maintained a very tight total cholesterol range from July, 2014 through October, 2014.

In November, 2014, Patient A was administered Crestor (40 mg) as a substitute for Lipitor (80 mg) and administered 3000 mg of Omega-3 fish oil(s). The total cholesterol decreased drastically to 103 mg/dL from a previously total cholesterol value of 136 mg/dL, a decrease of approximately 24%. The previous medicaments were the same as illustrated in Table 2. Also, Patient A experienced a root canal extraction. There was no problem in the repair of the root canal; however, Patient A experienced no pain for months while the roots were decaying, a result of vitamin D therapy.

### Example 13: Experiment 12

In December, 2014, Patient A continued his administration of Crestor and Zetia, but he added to his medicaments Curcumin, quercetin, bacopa, bioperine and phosphatidylcholine. There was a definite effect on dementia, short and long term memory.

### Example 14: Experiment 13

Attempting to find the lower concentration limit necessary to treat the autoimmune diseases psoriasis, keratosis and myopathy; vitamin D₃ was lowered to 1000 IU and Omega-3 fish oil(s) was lowered to 600 mg. During January, 2015, Patient A noticed a slight muscle pain in the buttocks and lower back. Vitamin D₃ measured in the blood as total vitamin D, 25-OH was 31.4 ng/ml.

Vitamin D₃ was increased to 2000 IU per day in February, 2015. The pain associated with myositis soon disappeared; however, due to the low concentration of Omega-3 (s) a small skin rash or seborrheic keratosis appeared. Early May, 2015 the Omega-3 fish oil(s) was increased to 1600 mg (600+1000 mg) whose EPA concentration was 847 mg and whose DHA concentration was 453 mg. After the medicament Omega-3 oil(s) was administered, the skin condition disappeared.

### Example 15: Experiment 14

At the end of April, 2015, Patient A's Omega oil(s) were increased to 2980 mg, but this time Omega 6 and Omega 9 oils were added as shown in Table 2.

Omega-3 oils were broken-down as: EPA-800 mg, DHA-400 mg, Omega 6-276 mg and Omega 9-170 mg. Within the next month, July, 2015, seborrheic keratosis returns and is believed due to the Omega 6 oil. Vitamin C and vitamin B complex were removed without any adverse effects to the disease patterns. The soft gum and bleeding gums were repaired where the gums were hard and no bleeding occurred.

For September, 2015, Omega 6 and Omega 9 were eliminated as medicaments and the seborrheic keratosis disappeared. Also vinpocetine at 30 mg was added as a medicament in June, 2015. The total cholesterol decreased by approximately ten percent, not a substantial amount; however, the total cholesterol tolerance interval is very narrow from June through August; hence, vinpocetine stabilizes cholesterol as well as lowers the total cholesterol. A result observed with Omega-3 fish oils. Again another organic compound found effective with a statin(s).

At a similar time, Patient B was given vinpocetine when she was having minor preictals every other day. Vinpocetine eliminated the minor auras. Patient A reported an enhancement of long and short memory with bacopa included.

### Example 16: Experiment 15

Later in August, 2015 and early September, Omega-3 oil(s) were removed as a medicament for Patient A. Within a few days, Patient A was covered entirely on the front right chest and on the right upper and lower back with pilaris keratosis. The Omega-3 oil(s) were introduced at a concentration of 2100 mg and the pilaris keratosis disappeared within 2 weeks; hence, vinpocetine will not treat the autoimmune disease of keratosis; however, it has a strong effect on controlling the total cholesterol after Omega-3 oil(s) has been administered as well as controlling dementia.

### Data Tables

Table 2 is an accumulation of Patient A's data for total cholesterol illustrated as total cholesterol (TC), high density lipoprotein (HDL), low density lipoprotein (LDL), triglycerides (TG), drug type and their concentrations and the comments associated with the drugs, vitamins, hormones, oils, fish oils, statins, organic compounds, vitamin D, vitamin D3 blood concentration, and adsorption parameters.

TABLE 2
Patient A Data

| | TC | HDL | LDL | TG | DRUG | CONC. | |
|---|---|---|---|---|---|---|---|
| DATE | (mg/dL) | (mg/dL) | (mg/dL) | (mg/dL) | type | (mg/day) | COMMENTS |
| May 2001 | | | | | | | quadrupel by-pass |
| | | | | | | | surgery |
| Jun. 2001 | | | | | 1, 34 | 40, 100 | Lipitor, Lopressor |
| Nov. 2001 | 187 | 47 | 123 | 105 | 1, 9 | 40, 50 | Lipitor, Metoprolol |
| Feb. 2003 | 170 | 53 | 90 | 134 | 1, 9, 16 | 40, 50, 1000 | Niacin |
| Mar. 2003 | 198 | 51 | 128 | 95 | 1, 9 | 40, 25 | |
| Nov. 2003 | 182 | 62 | 107 | 65 | 1, 9 | 40, 25 | |
| Feb. 2004 | 198 | 51 | 128 | 95 | 1 | 80 | Lipitor |
| Aug. 2004 | 129 | 54 | 63 | 60 | 1 | 80 | |
| Mar. 2005 | 183 | 37 | 62 | 417 | 1 | 80 | |
| May 2005 | 156 | 51 | 92 | 64 | 1, 2 | 80, 145 | Lipitor, Tricor |
| Jan. 2006 | 108 | 39 | 57 | 59 | 1, 2 | 80, 145 | Detoxification cleaning |
| Apr. 2006 | 161 | 55 | 91 | 75 | 1, 2 | 80, 145 | |
| Nov. 2006 | 160 | 54 | 90 | 78 | 3 | 10, 40 | Vytorin |
| | | | | | | | (Zetia/Simuvastatin) |
| Jun. 2007 | 149 | 50 | 86 | 63 | 3 | 10, 40 | |
| Sep. 2008 | 147 | 35 | 80 | 161 | 3 | 10, 40 | |
| Dec. 2008 | 225 | 40 | 166 | 94 | 4 | 80 | Provacol |
| Mar. 2009 | 211 | 50 | 137 | 118 | 4, 5 | 80, 10 | Provachol, Zetia |
| Jun. 2009 | 176 | 38 | 110 | 140 | 4 | 80 | Provachol |
| Aug. 2009 | 175 | 42 | 114 | 97 | 1, 2 | 40, 145 | Lipitor, Tricor |
| Jan. 2010 | 138 | 51 | 76 | 56 | 1, 5, 10 | A, B | Lipitor, Zetia, |
| | | | | | | | Lisinopril, Digoxin, |
| | | | | | | | Warfarin, Metoprolo |
| Mar. 2010 | 116 | 40 | 63 | 76 | 1, 5, 10 | A, B | |
| Apr. 2010 | | | | | 23, 35 | A, B, 240 | Atrial fibrillation, |
| | | | | | | | defibrillator implanted |
| Feb. 2011 | 160 | 43 | 92 | 125 | 1, 5, 10 | A, B | psoriasis on legs |
| Aug. 2011 | 190 | 42 | 107 | 203 | 3, 10 | 10, 40, B | psoriasis on legs, back, |
| | | | | | | | Nov. 2011 Lasik |
| Mar. 2012 | 151 | 40 | 85 | 129 | 23, 19 | A, B, 80 | psoriasis on legs, back, |
| | | | | | | | scalp |
| Sep. 2012 | 139 | 36 | 73 | 148 | 23, 19 | A, B, 80 | psoriasis on legs, back, |
| | | | | | | | scalp, ears and on |
| | | | | | | | Feb. 2013 appears on face |
| Mar. 2013 | 116 | 33 | 61 | 112 | 22, 11, 12, | C, 3000 IU, | 2-weeks itching gone, |
| | | | | | 13, 14 | 125, 1000, | no bleeding from skin, |
| | | | | | | 1000 | no psoriasis, no |
| | | | | | | | myopathy, 2-weeks |
| | | | | | | | no gum bleeding, EPA-667, |
| | | | | | | | DHA-333 |
| May 2013 | 133 | 36 | 73 | 118 | 22, 11, 12, | C, 3000 IU, | |
| | | | | | 13, 14 | 125,1000, | |
| | | | | | | 1000 | |
| Jun. 2013 | 127 | 36 | 56 | 176 | 22, 11, 12, | C, 3000 IU, | no psoriasis, no |
| | | | | | 13, 14 | 125, 1000, 0 | myopia, no vitamin C |
| Jul. 2013 | 132 | 36 | 77 | 96 | 22, 11a, 12, | C, 6000 IU, | no psoriasis, no |
| | | | | | 13,14 | 125, 1000, 0 | myopia, no vitamin C |
| Aug. 2013 | 138 | 36 | 75 | 134 | 22, 11a, 12, | C, 5000 IU, | psoriasis returns, there |
| | | | | | 13, 14 | 125, 0, 1000 | is a new skin disease, |
| | | | | | | | Keratosis, small pilaris |
| | | | | | | | keratosis on back, left |
| | | | | | | | head, Total vitamin D, |
| | | | | | | | 25-OH is 44.6 ng/ml |
| Sep. 2013 | 158 | 41 | 88 | 143 | 22, 11a, 12, | C, 12,000 IU, | no psoriasis, seborrheic |
| | | | | | 13, 14 | 125, | keratosis takes months |
| | | | | | | 2000, 1000 | before it will go away. |
| | | | | | | | Seen on rt. arm. Old |
| | | | | | | | skin defect. Everything |
| | | | | | | | back to normal. Total |
| | | | | | | | vitamin D, 25-OH is |
| | | | | | | | 89.3 ng/ml EPA-1334, |
| | | | | | | | DHA-666 |
| Oct. 2013 | 132 | 40 | 72 | 98 | 22, 11, 12, | C, 4000 IU, | |
| | | | | | 13, 14 | 125, 2000, | |
| | | | | | | 1000 | |
| Nov. 2013 | 150 | 43 | 79 | 139 | 22, 15, 12, | C, 1000 IU | D3-Nature's Made |
| | | | | | 13, 14 | powder and | powder myopathy |
| | | | | | | 1000 IU from | returned slightly, no |
| | | | | | | Omega-3, | psoriasis, no keratosis. |
| | | | | | | 125, 1000, | total vitamin D, 25-OH |
| | | | | | | 1000 | is 64.1 ng/ml |
| Dec. 2013 | 151 | 40 | 87 | 120 | 22, 15, 12, | C, 1000 IU | no psoriasis, no |
| | | | | | 13, 14 | powder and | keratosis, total vitamin |
| | | | | | | 1000 IU from | D, 25-OH is 35 ng/ml |
| | | | | | | Omega-3, | |
| | | | | | | 125, 1000, | |
| | | | | | | 1000 | |
| Jan. 2014 | 176 | 39 | 96 | 207 | 3, 10, 15, | 10/40, B, | no psoriasis, no |
| | | | | | 12, 13, 14 | 1000 IU from | keratosis, have split |
| | | | | | | Omega-3, | vytorin to10/40 |
| | | | | | | 125, 1000, | Ezetimbe/Simvastatin; |
| | | | | | | 1000 | myopathy returns-very |
| | | | | | | | high pain level, 8 of 10 |
| Feb. 2014 | 186 | 48 | 105 | 164 | 3, 10, 11, | 10/40, B, | no psoriasis, no |
| | | | | | 12, 13, 14 | 3000 IU, | keratosis, no myopathy, |
| | | | | | | 125, 1000, | niacin lowers |
| | | | | | | 1000 | cholesterol, total |
| | | | | | | | vitamin D, 25-OH is |
| | | | | | | | 33.2 ng/ml |
| Mar. 2014 | 163 | 41 | 90 | 160 | 3, 10, 11, 12, | 10/40, B, | no psoriasis, no |
| | | | | | 13, 14, 16 | 3000 IU, | keratosis, no myopathy, |
| | | | | | | 125, 1000, | niacin lowers |
| | | | | | | 1000, 500 | cholesterol, total |
| | | | | | | | vitamin D, 25-OH |
| | | | | | | | is 33.2 ng/ml |
| Apr. 2014 | 137 | 46 | 60 | 157 | 17, 5, 10, 11, | 40, 10, B, | |
| | | | | | 12, 13, 14 | 3000 IU, 125, | |
| | | | | | | 1000, 1000 | |
| May 2014 | 109 | 40 | 44 | 124 | 17, 5, 10, 11, | 40, 10, B, | conc. of fish oil 3X and |
| | | | | | 12, 13, 14 | 5000 IU, 125, | EPA and DHA is 3X |
| | | | | | | 3000, 1000 | |
| Jun. 2014 | 116 | 43 | 52 | 103 | 17, 5, 10, 11, | 40, 10, B, | |
| | | | | | 12, 13, 14 | 5000 IU, 125, | |
| | | | | | | 3000, 1000 | |
| Jul. 2014 | 142 | 46 | 68 | 138 | 22, 11, 12, | C, 3000 IU, | total vitamin D, 25-OH |
| | | | | | 13, 14 | 125, 1000, | is 47.3 ng/ml |
| | | | | | | 1000 | |
| Aug. 2014 | 150 | 40 | 70 | 199 | 22, 11, 12, | C, 5000 IU, | |
| | | | | | 13, 14, 18 | 125, 3000, | |
| | | | | | | 1000, 20 | |
| Sep. 2014 | 157 | 42 | 85 | 148 | 22, 11, 12, | C, 5000 IU, | |
| | | | | | 13, 14, 18 | 125, 3000, | |
| | | | | | | 1000, 20 | |
| Oct. 2014 | 136 | 42 | 72 | 110 | 22, 11, 12, | C, 3000 IU, | |
| | | | | | 13, 14, 18 | 125, 1000, | |
| | | | | | | 1000, 20 | |
| Nov. 2014 | 103 | 38 | 45 | 99 | 17, 5, 10, 11, | 40, 10, B, | bioperine has no effect |
| | | | | | 12, 13, 14 | 5000 IU, 125, | on cholesterol or |
| | | | | | | 3000, 1000 | psoriasis, keratosis, |
| | | | | | | | gum disease or |
| | | | | | | | myopathy, root canal, |
| | | | | | | | no pain |
| Dec. 2014 | 118 | 36 | 48 | 171 | 17, 5, 10, 11, | 40, 10, B, | taking curcumin, |
| | | | | | 12, 13, 14 | 5000 IU, 125, | quercetin, bacopa, |
| | | | | | | 3000, 1000 | bioperine and |
| | | | | | | | phosphatidyl cholin had |
| | | | | | | | no effect on cholesterol, |
| | | | | | | | but a definite effect on |
| | | | | | | | dementia, short and |
| | | | | | | | long term memory |
| | | | | | | | observed by Patient A |
| Jan. 2015 | 123 | 44 | 55 | 121 | 17, 5, 10, | 40, 10, B, | total vitamin D, 25-OH |
| | | | | | 11, 12, 32, | 1000 IU, 125, | is 31.4 ng/ml, slight |
| | | | | | 14 | 600, 1000 | muscle pain in buttocks & |
| | | | | | | | lower back, EPA- |
| | | | | | | | 180, DHA-120 |
| Feb. 2015 | 135 | 45 | 65 | 123 | 17, 5, 10, | 40, 10, B, | pain in lower back is |
| | | | | | 11,12, 32, | 2000 IU, 125, | gone however dry skin |
| | | | | | 14 | 600, 1000 | or keratosis returned |
| Apr. 2015 | 134 | 46 | 56 | 162 | 17, 5, 10, | 40, 10, B, | common cold-increase |
| | | | | | 11, 12, 13 | 3000 IU, 125, | vitamin C to |
| | | | | | and 32, 14 | 1600 mg, | 8000 mg/day- (3) days, |
| | | | | | | 1000 | EPA-847, DHA-453 no |
| | | | | | | | pain, no skin condition |
| Apr. 2015 | 121 | 43 | 53 | 127 | 17, 5, 10, | 40, 10, B, | EPA-1247, DHA-653 |
| | | | | | 11, 12, 13, | 3000 1U, 125, | |
| | | | | | 32 and 33, | 2650 mg, | |
| | | | | | 14 | 1000 | |
| Jun. 2015 | 108 | 43 | 45 | 100 | 17, 5, 10, | 40, 10, B, | Omega-3 1484, EPA |
| | | | | | 11, 27 and | 2000 IU, | 465, DHA 252, Omega |
| | | | | | 33, 25, 26 | 1484, 30, | 6-276, Omega 9-170, |
| | | | | | | 1200 | vinpocetine, |
| | | | | | | | phosphatidyl choline, |
| | | | | | | | no vitamin C, no |
| | | | | | | | vitamin B |
| Jul. 2015 | 113 | 43 | 45 | 124 | 17, 5, | 40, 10, B, | Seborrheic keratosis |
| | | | | | 10, 11, 33, | 2000 IU, | returned due to Omega |
| | | | | | 25, 26 | 1050, 30, | 6 or Omega 9.. |
| | | | | | | 1200 | |
| Aug. 2015 | 107 | 33 | 44 | 150 | 17, 5, | 40, 10, B, | elimination of Omega 6 |
| | | | | | 10, 11, 33, | 2000 IU, | and Omega 9, |
| | | | | | 25, 26 | 1050, 30, | eliminated seborrheic |
| | | | | | | 1200 | keratosis, vinpocetine, |
| | | | | | | | stabilizes cholesterol |
| | | | | | | | and lowers it as well as |
| | | | | | | | inhibits preictal events, |
| | | | | | | | Patient B, and enhances |
| | | | | | | | long and short term |
| | | | | | | | memory with Bacopa |
| | | | | | | | included |

Table 3 is an accumulation of Patient B's data for total cholesterol illustrated as total cholesterol (TC), high density lipoprotein (HDL), low density lipoprotein (LDL), triglycerides (TG), drug type and their concentrations and the comments associated with the drugs, vitamins, hormones, oils, fish oils, statins, organic compounds, vitamin D, vitamin D3 blood concentration, and adsorption parameters.

TABLE 3
Patient B Data

| | TC | HDL | LDL | TG | DRUG | CONC. | |
|---|---|---|---|---|---|---|---|
| DATE | (mg/dL) | (mg/dL) | (mg/dL) | (mg/dL) | type | (mg/day) | COMMENTS |
| Jan. 1998 | 218 | 101 | 99 | 106 | | | |
| Jul. 2001 | 217 | 105 | 104 | 48 | | | |
| Jun. 2003 | 246 | 97 | 137 | 57 | | | May 23, 2003 Santa Fe |
| | | | | | | | trip - memory |
| | | | | | | | loss |
| Oct. 2004 | 259 | 110 | 136 | 64 | 1 | | Sep. 26 2004 seizure |
| | | | | | | | hospital, Oct 4, 2004 |
| | | | | | | | seizure |
| Dec. 2004 | 174 | 91 | 72 | 53 | 1 | 500 | Nov. 31, 2004 seizure |
| | | | | | | | hospital, |
| | | | | | | | detoxification |
| Apr. 2005 | 156 | 87 | 61 | 41 | 1 | 750 | Jan. 3, 2005 seizure, |
| | | | | | | | detoxification |
| May 2005 | 207 | 84 | 110 | 64 | 1 | 1000 | |
| Mar. 2006 | 199 | 96 | 90 | 64 | 1 | 1000 | |
| Jun. 2006 | 190 | 78 | 100 | 60 | 1 | 1000 | |
| Oct. 2006 | 218 | 86 | 119 | 63 | 1 | 1500 | Oct. 30, 2006 seizure |
| | | | | | | | 45 minutes |
| Mar. 2007 | 211 | 95 | 103 | 66 | 1, 2 | 1500, 1000 | Jul. 24 2007 strange |
| | | | | | | | feeling on & off |
| | | | | | | | all day |
| Jan. 2008 | 209 | 90 | 100 | 95 | 1, 2 | 1500, 1500 | Sep. 14 2007 auras, |
| | | | | | | | May 5, 2008 speech |
| | | | | | | | impaired, |
| | | | | | | | Nov. 25, 2009 seizure, |
| Sep. 2010 | 208 | 107 | 83 | 108 | 1, 2, 12 | 1500, 2000, | Jan. 21, 2009 seizure, |
| | | | | | | D | Aug. 29, 2009 speech |
| | | | | | | | impaired, Mar. 2010 |
| | | | | | | | calcuim and |
| | | | | | | | vitamin D. Total |
| | | | | | | | vitamin D, 25- |
| | | | | | | | OH is 37 ng/ml |
| Jun. 2012 | 240 | 103 | 121 | 79 | 1, 2, 10 | 1500, 2500, | Jan. 6, 2010 (4) |
| | | | | | | 1200,600, | seizures, |
| | | | | | | 1000 IU | hospital, Feb. 28, 2011 |
| | | | | | | | seizures, Aug. 1, 2012 |
| | | | | | | | speech impaired, |
| | | | | | | | Jun. 27, 2012 total |
| | | | | | | | vitamin D, 25- |
| | | | | | | | OH is 79 ng/ml |
| Aug. 2013 | 179 | 88 | 80 | 55 | 1, 2, 3, 4, | 1500, 3000, | Omega-3 at 2100 |
| | | | | | 5, 10 | A, E | mg, vitamin D₃ |
| | | | | | | | at 1000 IU |
| Sep. 2013 | 198 | 94 | 85 | 93 | 13 | F | Omega-3 at 2100 |
| | | | | | | | mg, vitamin D₃ |
| | | | | | | | at 3000 IU |
| Oct. 2013 | 195 | 96 | 86 | 66 | 13 | F | |
| Dec. 2013 | 205 | 116 | 72 | 83 | 13 | F | |
| Feb. 2014 | 221 | 128 | 79 | 70 | 13 | F | Feb. 28, 2014 aura, |
| | | | | | | | can't speak, |
| | | | | | | | lasted 10 min. |
| Mar. 2014 | 195 | 118 | 69 | 41 | 13 | F | |
| Apr. 2014 | 237 | 132 | 94 | 54 | 13 | F | |
| May. 2014 | 231 | 112 | 105 | 69 | 13 | F | |
| Jun. 2014 | 212 | 116 | 86 | 50 | 10, 11, | G, E, 100 | |
| | | | | | 14 | | |
| Jul. 2014 | 179 | 85 | 70 | 120 | 10, 11, | G, E, 100 | |
| | | | | | 14 | | |
| Aug. 2014 | 227 | 134 | 81 | 61 | 10, 11, | G, E, 100 | |
| | | | | | 14 | | |
| Sep. 2014 | 229 | 117 | 92 | 101 | 10, 11, | G, E, 100 | |
| | | | | | 14 | | |
| Jan. 2015 | 188 | 144 | 33 | 57 | 8, 10, 11, | G, E, 100, 20 | Crestor 20 mg. |
| | | | | | 14 | | aura |
| Feb. 2015 | 179 | 134 | 32 | 66 | 8, 10, 11, | G, E, 100, 20 | aura. total |
| | | | | | 14 | | vitamin D, 25- |
| | | | | | | | OH is 56.6 ng/ml |
| Apr. 2015 | 173 | 105 | 57 | 56 | 8, 10, 11, | G, E, 100, | aura |
| | | | | | 14, 15, | 20, H | |
| | | | | | 16, 17 | | |
| Apr. 2015 | 179 | 135 | 34 | 50 | 8, 10, 11, | G, E, 100, | increase |
| | | | | | 14, 15, | 20, H | frequency of |
| | | | | | 16, 17 | | auras, every |
| | | | | | | | other day |
| May 2015 | | | | | 10, 11, | G, E, 100, H | no Crestor, |
| | | | | | 14, 15, | | increase |
| | | | | | 16, 17 | | frequency of |
| | | | | | | | auras, every |
| | | | | | | | other day |
| Jun. 2015 | | | | | 10, 11, | G, E, 100, I | total vitamin D, |
| | | | | | 14, 15, | | 25-OH is 44.2 |
| | | | | | 16, 17, | | ng/ml, no auras, |
| | | | | | 18, 19 | | vinpocetine and |
| | | | | | | | coenzyme Q10 |
| | | | | | | | (trans form) |
| | | | | | | | inhibits preictals |

Table 4 describes the drugs and organic compounds administered to Patient A as well as the concentrations of each drug and organic compound.

TABLE 4
Patient A Drug Types and Doses

| Number/ | | | Concentrations |
|---|---|---|---|
| Letter | Drug | Organic Compounds | (mg/day) |
| 1 | Lipitor | | |
| 2 | Tricor | | 145 |
| 3 | Vytorin | | 10/40 |
| 4 | Provachol | | 80 |
| | (Pravastatin) | | |
| 5 | Zetia | | 10 |
| 6 | Lisinopril | | 5 |
| 7 | Digoxin | | 0.25 |
| 8 | Warfarin Sodium | | 3.04 |
| 9 | Metoprolol | | 200 |
| 10 | combination of 6, 7, | | 5, 0.25, 3.04, 200 |
| | 8 & 9 | | |
| 11 | | vitamin D₃, 2000 IU, | 2000 IU |
| | | Carlson (soft gels) | |
| 11a | | vitamin D₃, 5000 IU, | 5000 IU |
| | | Nature's Bounty | |
| 12 | | B mega B-125, Ultra | 1 capsules |
| | | Plan | |
| 13 | | Omega-3 fish oil 1000 | 1000 mg EPA-667/DHA- |
| | | with vitamin D₃ 1000 | 333 |
| | | IU, Optim Nutrition | |
| 14 | | vitamin C, Vitacost 1000 | |
| 15 | | vitamin D₃, 1000 IU, | 1000 IU |
| | | Nature's Made (tablets) | |
| 16 | | Niacin (B-3), Twin Lab | 500 |
| 17 | Crestor | | 40 |
| 18 | | Bioperine | 20 |
| 19 | Lasix | | 80 |
| 20 | Simvastatin | | 40 |
| 21 | | combination of 10 & 19 | 5, 0.25, 3.04, 200, 80 |
| 22 | | combination of 1, 5 & 21 | 80, 10, 5, 0.25, 3.04, |
| | | | 200, 80 |
| 23 | | combination of 1, 5 &10 | 80, 10, 5, 0.25, 3.04, |
| | | | 200 |
| 24 | | Aspirin, Kirkland | 325 |
| 25 | | Vinpocetine, Vitacost | 30 |
| 26 | | Phosphatidyl Choline | 1200 |
| | | complex, Country Life | |
| 27 | | Triple Omega, Omega- | Omega-3-434, Omega- |
| | | 3-6-9, Nature Made | 6-276, Omega-9-170 |
| 28 | | Curcumin 2K, Stop | 1330 |
| | | Aging Now | |
| 29 | | Quercetin, Swanson | 650 |
| 30 | | Bacopa, Nature's | 500 |
| | | Answer | |
| 31 | | Max-Q10, Stop Aging | 200 |
| | | Now | |
| 32 | | Omega-3 fish oils with | 600 fish oil EPA |
| | | vitamin E, Ultra Plan | 180/DHA120 |
| 33 | | Mega EFA, Omega-3 | 2100 mg fish oil, EPA |
| | | EPA & DHA, Vitacost | 800/DHA 400 |
| 34 | Lopressor | | 100 |
| 35 | Diltiazem | | 240 |
| A | | concentration of 1 & 5 | 80 & 10 |
| B | | concentration of 10 | 5, 0.25, 3.04, 200 |
| C | | concentration of A & 21 | 80, 10, 5, 0.25, 3.04, |
| | | | 200, 80 |

Table 5 describes the drugs and organic compounds administered to Patient B as well as the concentrations of each drug and organic compound.

TABLE 5
Patient B Drug Types and Doses

| Number/ | | | Concentrations |
|---|---|---|---|
| Letter | Drug | Organic Compounds | (mg/day) |
| 1 | Depakote | | |
| 2 | Keppra | | |
| 3 | | Mega EFA, Omega-3 | 2100 mg fish oil, EPA- |
| | | EPA & DHA, Vitacost | 800, DHA-400 |
| 4 | | vitamin C, 1000, Vitacost | 2000 |
| 5 | | vitamin E-400 IU, Nature | 400 IU |
| | | Bounty | |
| 6 | | vitamin D3, 2000 IU, | 2000 IU |
| | | Vitacost | |
| 7 | | vitamin D3, 5000 IU, | 5000 IU |
| | | Vitacost | |
| 8 | Crestor | | 20 |
| 9 | | Mega-B-125, Ultra Plan | 125 |
| 10 | | Calcium Magnesium | 1200, 600, 1000 IU |
| | | Citrate with vitamin D3, | |
| | | Solgar | |
| 11 | | Liquid CoQ10, Qunol | 100 |
| 12 | | Calcium, Magnesium, | 1600, 800, 400 IU |
| | | vitamin D, Complete | |
| | | Bone, Rx Formulations | |
| 13 | | concentration of 1, 2, 3, | 1500, 3000, 2100, |
| | | 4, 5,6, 9, 10 | 2000, 400 IU, 2000 IU, |
| | | | 125, 1200, 600, |
| | | | 1000 IU |
| 14 | | combination of 1, 2, 3, 4, | 1500, 3000, 2100, |
| | | 5, 7, 9 | 2000, 400 IU, 5000 IU, |
| | | | 125 |
| 15 | | Curcumin 2K with | 1330, 20 |
| | | BioPerine, Stop Aging | |
| | | Now | |
| 16 | | Quercetin, Swanson | 650 |
| 17 | | Bacopa, Nature's Answer | 500 |
| 18 | | Vinpocetine, Vitacost | 30 |
| 19 | | Coenzyme Q10, Stop | 200 |
| | | Aging Now | |
| A | | concentration of 3, 4, 5 | 2100, 2000, 400 IU |
| B | | concentration of 3, 4, 5, | 4200, 2000, 400 IU, |
| | | 6, 9 | 2000 IU, 125 |
| C | | concentration of 3, 4, 5, | 4200, 2000, 400 IU, |
| | | 7, 9 | 5000 IU, 125 |
| D | | concentration of 12 | 1600 Ca, 800 Mg, |
| | | | 400 IU, D-3 |
| E | | concentration of 10 | 1200, 600, 1000 IU |
| F | | concentration of 13 | 1500, 3000, 2100, |
| | | | 2000, 400 IU, 2000 IU, |
| | | | 125, 1200, 600, |
| | | | 1000 IU |
| G | | concentration of 14 | 1500, 3000, 2100, |
| | | | 2000, 400 IU, 5000 IU, |
| | | | 125 |
| H | | concentration of 15, 16, | 1330, 20, 650, 500 |
| | | 17 | |
| I | | concentration of 15, 16, | 1330, 20, 650, 500, 30, |
| | | 17, 18, 19 | 200 |

Table 6 describes the Detox Schedule for Patient A (see also FIG. 1).

TABLE 6
Detox Schedule

| Hour | Product | Amount | In Liquid |
|---|---|---|---|
| 7:00 AM | Greens First | 1-2 scoops + HMF | 8 oz water |
| | | (acidophilus) | |
| | | powder/superpowder | |
| | | 1/2-1 tsp | |
| 8:00 AM | Ultra Sustaine | 2 scoops | 8 oz Rice Dream or |
| | | | Cranberry with water |
| | or Permavite | 1-1/2 Tbls | 8 oz Rice Dream or |
| | | | Cranberry with water |
| 8:30 AM | water | 2 cups (16 oz) | water |
| | MetaFiber | 2 scoops | 8 oz Cranberry with |
| | | | water |
| | or Ground Flax | 1/4 cup | 8 oz Cranberry with |
| | Seeds | | water |
| 9:00 AM | water | 2 cups (16 oz) | water |
| | Ultra Clear or Plus | 2 scoops | 8 oz Rice Dream |
| 12:00 PM | Greens First | 1-2 scoops + HMF | at least 8 oz water |
| | | (acidophilus) | |
| | | powder/superpowder | |
| | | 1/2-1 tsp | |
| 1:30 PM | water | water | 2 cups (16 oz) |
| | Ultra Clear or Plus | 2 scoops | 8 oz Rice Dream |
| 3:30 PM | water | 2 cups (16 oz) | water |
| | Ultra Sustaine | 2 scoops | 8 oz Rice Dream |
| | or Permavite | 1-1/2 Tbls | 8 oz Rice Dream |
| 5:30 PM | water | 2 cups (16 oz) | water |
| | Ultra Clear or Plus | 2 scoops | 8 oz Rice Dream |
| 8:00 PM | MetaFiber | 2 scoops | 8 oz Cranberry with |
| | | | water |
| | or Ground Flax | 1/4 cup | 8 oz Cranberry with |
| | Seeds | | water |
| 8:30 PM | Smooth Move Tea | 1 tea bag | 2 cups boiling water |
| | or Herbatox | 3-4 capsules | |

* Ultra Meal can be used throughout day when you feel hungry and to pick up your energy (2 scoops) to 8 oz liquid

Table 7 is a list of autoimmune diseases and the cellular and cytokine characteristics/markers that may be used to identify a patient having one or more of these conditions.

TABLE 7
Autoimmune Disease Markers

| Autoimmune Disease | Markers |
|---|---|
| Psoriasis | CD8+ T cells, HLA-CW6, IL-12b, IL-23b, TNF- |
| | alpha, NF-kB |
| Ankylosing Spondylitis | CD8, HLA-B27 |
| Antiphospholipid syndrome | HLA-DR7, HLA-B8, GKA-DR2, HLA-DR3 |
| Celiac | HLA-DQ8, DQ 2.5 |
| Crohn's | Th17, Th1, ATG16L1, CARD15, XBP1 |
| Diabetes mellitus I | HLA-DR3, HLA-DR4 |
| Mixed connective tissue | HLA-DR4 |
| Michan-Habermann | T cells |
| Multiple sclerosis | HLA-DR2, PECAM-1 |
| Myastlienia gravis | HA-B8, HLA-DR3, HLA-DR1 |
| Myositis, dermatomyositis Polymyositis | IFN-gama, IL-1, TNF-alpha |
| Psoriatic arthritis | HLA-B27 |
| Rheumatoid arthritis | HLA-DR4, PTPN22, B cells, TNF alpha, IL-17, |
| | IL-1, IL-6, IL-15 |
| Spondyloarthropathy | HLA-B27 |
| undifferentiated connective tissue | HLA-DR4 |
| autoimmune Uveitis | HLA-B27 |

Table 8 illustrates the various fatty acids and their concentrations as fatty acid triglycerides and fatty acid ethyl esters measured from the blood serum of Patient A. Also shown are the concentrations of vitamin D₃, as cholecalciferol D₃ in its powder and gel forms as well as the total vitamin D and 25-OH.

TABLE 8
Patient A Treatment Data

| | TC | EPA | DHA | 25-OF | |
|---|---|---|---|---|---|
| DATE | (mg/dL) | (mg) | (mg) | (ng/ml) | COMMENTS |
| 2/2013 | 116 | 667 | 333 | | triglyceride-Omega-3 |
| 5/2013 | 133 | 667 | 333 | | triglyceride-Omega-3 |
| 6/2013 | 127 | 667 | 333 | | triglyceride-Omega-3 |
| 7/2013 | 132 | 667 | 333 | | triglyceride-Omega-3 |
| 8/2013 | 138 | 0 | 0 | 44.6 | triglyceride-Omega-3 |
| 9/2013 | 158 | 1334 | 666 | 89.3 | triglyceride-Omega-3 |
| 10/2013 | 132 | 1334 | 666 | | |
| 11/2013 | 150 | 667 | 333 | 64.1 | triglyceride-Omega-3 |
| 12/2013 | 151 | 667 | 333 | 35 | triglyceride-Omega-3 |
| 1/2014 | 176 | 667 | 333 | | triglyceride-Omega-3 |
| | | | | | 1000 IU from Omega-3 |
| | | | | | significant pain from |
| | | | | | vytorin |
| 2/2014 | 186 | 667 | 333 | 33.2 | 3000 IU, no myopathy, |
| | | | | | vytorin |
| 3/2014 | 163 | 667 | 333 | 33.2 | 3000 IU, no myopathy, |
| | | | | | vytorin |
| 4/2014 | 137 | 667 | 333 | | 3000 IU, Crestor, Zetia |
| 5/2014 | 109 | 2001 | 999 | | 5000 IU, Crestor, Zetia |
| 6/2014 | 116 | 2001 | 999 | | 5000 IU, Crestor, Zetia |
| 7/2014 | 142 | 667 | 333 | 47.3 | 3000 IU, Lipitor, Zetia |
| 8/2014 | 150 | 2001 | 999 | | 5000 IU, Lipitor, Zetia |
| 9/2014 | 157 | 2001 | 999 | | 5000 IU, Lipitor, Zetia |
| 10/2014 | 136 | 667 | 333 | | 3000 IU, Lipitor, Zetia |
| 11/2014 | 103 | 2001 | 999 | | 5000 IU, Crestor, Zetia |
| 12/2014 | 118 | 2001 | 999 | | 5000 IU |
| 1/2015 | 123 | 180 | 120 | 31.4 | 1000 IV, slight pain |
| | | | | | lower back |
| 2/2015 | 135 | 180 | 120 | | 2000 IU, pain gone |
| 4/2015 | 134 | 847 | 453 | | 3000 IU, triglyceride |
| | | | | | fatty acids & fatty acid |
| | | | | | ethyl esters, 3000 IU, |
| | | | | | combination fatty acids |
| 4/2015 | 121 | 1247 | 653 | | Omega 6-276, Omega |
| | | | | | 9-170, vinpocetine, |
| | | | | | phosphatidyl choline |
| 6/2015 | 108 | 465 | 252 | | Seborrheic keratosis |
| | | | | | returned |
| 7/2015 | 113 | 667 | 333 | | Omega-3 fatty acid |
| | | | | | ethyl ester |
| 8/2015 | 107 | 667 | 333 | | eliminate Omega 6 and |
| | | | | | Omega 9, eliminated |
| | | | | | keratosis |

TABLE 9
A copy of the Lipid-modifying Effects 10 mg and
20 mg Crestor (AstraZeneca Pharmaceuticals) in Primary
Dysbetalipoproteinemia (Type III
hyperlipoproteinemia) Change (95% C1)
from Baseline (N = 32).

| | | Median | Median |
|---|---|---|---|
| | | percent change | percent change |
| | | from baseline | from baseline |
| | Median at | (95% C1 | (95% C1 |
| | Baseline | CRESTOR | CRESTOR |
| Marker | (mg/dL) | 10 mg) | 20 mg |
| Total-C | 342.5 | −43.3 | −47.6 |
| Triglycerides | 503.5 | −40.1 | −43 |
| NonHDL-C | 294.5 | −48.2 | −56.4 |
| VLDL + IDL-C | 209.5 | −46.8 | −56.2 |
| LDL-C | 112.5 | −54.4 | −57.3 |
| HDL-C | 35.5 | 10.2 | 11.2 |
| RLP-C | 82 | −56.4 | −64.9 |
| Apo-E | 16 | −42.9 | −42.5 |

What is claimed is:

1. A pharmaceutical composition for the treatment of a disease or disorder comprising i) at least one fatty acid, and ii) at least one cholesterol lowering compound.

2. A pharmaceutical composition for reducing total cholesterol and maintaining total cholesterol homeostasis comprising i) at least one fatty acid, and ii) at least one cholesterol lowering compound.

3. The pharmaceutical composition of claim 2, wherein maintaining total cholesterol homeostasis comprises maintaining a total cholesterol percent relative range of about 15% between a maximum total cholesterol level and a minimum total cholesterol level.

4. The pharmaceutical composition of claim 2, wherein maintaining total cholesterol homeostasis comprises maintaining a total cholesterol percent relative range of about 10% between a maximum total cholesterol level and a minimum total cholesterol level.

5. The pharmaceutical composition of claim 2, wherein maintaining a total cholesterol homeostasis comprises maintaining a total cholesterol percent relative range of about 5% between a maximum total cholesterol level and a minimum total cholesterol level.

6. The pharmaceutical composition of claim 2, wherein maintaining total cholesterol homeostasis comprises maintaining a total cholesterol relative range of about 5 mg/dL to about 25 mg/dL between a maximum total cholesterol level and a minimum total cholesterol level.

7. The pharmaceutical composition of claim 2, wherein maintaining a total cholesterol homeostasis comprises maintaining a total cholesterol relative range of about 10 mg/dL to about 15 mg/dL between a maximum total cholesterol level and a minimum total cholesterol level.

8. The pharmaceutical composition of claim 2, wherein a maximum total cholesterol level and a minimum total cholesterol level are maintained below 200 mg/dL.

9. The pharmaceutical composition of claim 2, wherein a maximum total cholesterol level and a minimum total cholesterol level are maintained below 175 mg/dL.

10. The pharmaceutical composition of claim 2, wherein a maximum total cholesterol level and a minimum total cholesterol level are maintained below 150 mg/dL.

11. The pharmaceutical composition of claim 2, wherein total cholesterol is reduced at least 5%.

12. The pharmaceutical composition of claim 2, wherein total cholesterol is reduced at least 10%.

13. The pharmaceutical composition of claim 2, wherein total cholesterol is reduced at least 15%.

14. The pharmaceutical composition of claim 2, wherein total cholesterol is reduced at least 20%.

15. The pharmaceutical composition of claim 2, wherein total cholesterol is reduced at least 40%.

16. The pharmaceutical composition of claim 1, wherein the at least one fatty acid is a fatty oil, a fatty acid ethyl ester, a fatty acid triglyceride, a saturated acid, an oil, an ester or a triglyceride, or a combination thereof.

17. The pharmaceutical composition of claim 1, wherein the at least one fatty acid is an Omega-3 fatty acid, an Omega-6 fatty acid, an Omega-9 fatty acid, or a derivative thereof.

18. The pharmaceutical composition of claim 1, wherein the at least one cholesterol lowering compound is an HMG-CoA reductase inhibitor or statin, Ezetimibe, a fibrate, a carboxylic acid, Benezafibrate, Ciprofibrate, Gemfbroizil, Fenofibrate, Clinofibrate, niacin, bile acid sequestrants, Colestipol, Cholestyramine, Endur-Acin, Colesevelam, a PCSK9 enzyme inhibitor, or any combination thereof.

19. The pharmaceutical composition of claim 18, wherein the at least one HMG-CoA reductase inhibitor or statin is Atorvastatin, Fluvastatin, Lovastatin, Pitavastatin, Pravastatin, Rosuvastatin, Simvastatin, or any combination thereof.

20. The pharmaceutical composition of claim 1, further comprising at least one vitamin or hormone.

21. The pharmaceutical composition of claim 20, wherein the at least one vitamin is vitamin B, vitamin C or vitamin D, or a combination thereof.

22. The pharmaceutical composition of claim 21, wherein the at least one vitamin D is vitamin D₂ or vitamin D₃.

23. The pharmaceutical composition of claim 21, wherein the at least one vitamin C is ascorbate or ascorbic acid.

24. The pharmaceutical composition of claim 1, further comprising at least one organic compound, herb or derivative thereof.

25. The pharmaceutical composition of claim 24, wherein the at least one organic compound, herb or derivative thereof is bacopa, vinpocetine, an alkaloid, reserpine, reserpinine, akuammicine, majdine, vinerine, ervine, vineridine, tombozine, vincananine, vincanidine, vincamore, apovincamine, vincamore, apovincamine, or vincaminol.

26. The pharmaceutical composition of claim 1, wherein the disease or disorder is an autoimmune disease, an inflammatory disorder, a neurodegenerative disorder, a bacterial infection or a viral infection.

27. The pharmaceutical composition of claim 1, wherein the disease or disorder is psoriasis, keratosis, spongy gum and bleeding gum disease, atherosclerosis, heart disease, myopathy, neuropathy, common cold, myositis, arthritis, dementia, Parkinson's disease, Alzheimer's disease.

28. The pharmaceutical composition of claim 27, wherein the keratosis is actinic, pilaris or seborrheic.

29. A pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA) and at least 10 mg of a cholesterol lowering composition.

30. The pharmaceutical composition of claim 29, wherein the composition is formulated for daily oral administration for use in the treatment of psoriasis.

31. The pharmaceutical composition comprising at least 1000 mg of a fish oil, at least 667 mg of a EPA, at least 333 mg of a DHA and at least 10 mg of a cholesterol lowering composition.

32. The pharmaceutical composition of claim 31, wherein the composition is formulated for daily oral administration for use in the treatment of keratosis, atherosclerosis, heart disease, or any combination thereof.

33. A pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA), at least 10 mg of a cholesterol lowering composition and at least 1000 mg of vitamin C.

34. The pharmaceutical composition of claim 33, wherein the composition is formulated for daily oral administration for use in the treatment of spongy gum and bleeding gum disease.

35. A pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA), at least 10 mg of a cholesterol lowering composition and at least 3000 mg of vitamin C.

36. The pharmaceutical composition of claim 35, wherein the composition is formulated for daily oral administration for use in the treatment of a bacterial infection, viral infection or the common cold.

37. The pharmaceutical composition of claim 36, wherein the common cold comprises a viral infection of a portion of the upper respiratory tract.

38. A pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA), at least 10 mg of a cholesterol lowering composition and at least 2000 IU of vitamin D.

39. The pharmaceutical composition of claim 38, wherein the composition is formulated for daily oral administration for use in the treatment of myopathy or neuropathy.

40. A pharmaceutical composition comprising at least 600 mg of a fish oil concentrate, at least 600 mg of a fish oil, at least 180 mg of an eicosapentaenoic acid (EPA), at least 120 mg of a docosahexaenoic acid (DHA), at least 10 mg of a cholesterol lowering composition, and at least 250 mg of bacopa.

41. The pharmaceutical composition of claim 40, further comprising at least 10 mg of vinpocetine.

42. The pharmaceutical composition of claim 40, wherein the composition is formulated for daily oral administration for use in the treatment of dementia, Parkinson's disease or Alzheimer's disease.

43. A method for the treatment of a disease or disorder in a subject in need thereof comprising administering a therapeutically effective amount of a pharmaceutical composition comprising i) at least one fatty acid, and ii) at least one cholesterol lowering compound, such that said disease or disorder is treated
